(19) United States
(12) Patent Application Publication
(10) Pub. No.: US 2026/0298941 A1
(43) Pub. Date: Oct. 1, 2026

(54) METHODS FOR DETECTING A NEUTRALIZING ANTIBODY (NAB) AGAINST AN ANTI-TNF ALPHA ANTIBODY

(71) Applicant: Xentria, Inc., Chicago, IL (US)

(72) Inventors: Thomas Matthews, Chicago, IL (US); Kenneth Swart, Durham, NC (US)

(21) Appl. No.: 19/490,870

(22) PCT Filed: Jun. 7, 2024
(86) PCT No.: PCT/US2024/032915

§ 371 Date: Dec. 5, 2025

Related U.S. Application Data
(60) Provisional application No. 63/506,911, filed on Jun. 8, 2023.

(51) Int. Cl. G01N 33/68; G01N 33/543; G01N 33/58

(52) U.S. Cl. CPC G01N 33/6854; G01N 33/54326; G01N 33/581; G01N 33/582

(57) ABSTRACT

The present technology comprises methods for detecting a neutralizing antibody (NAb) against a target antibody. In some embodiments, the target antibody comprises an anti-Tumor Necrosis Factor-alpha (TNFα) antibody. In some embodiments, the target antibody comprises SEQ ID NO: 9-11 or 15-17 and SEQ ID NOs: 12-14 or 18-20. The present technology further comprises kits for performing the methods of the present technology.

### CROSS-REFERENCE TO RELATED APPLICATION(S)

This application claims the benefit of U.S. Provisional Patent Application No. 63/506,911, filed Jun. 8, 2023. The contents of this provisional applications are incorporated by reference in their entirety.

### SEQUENCE LISTING

This application contains an ST.26 compliant Sequence Listing, which was submitted in xml format via Patent Center and is hereby incorporated by reference in its entirety. The .xml copy, created on May 17, 2024, is named 140505-8006WO00_SL.xml and is 20,939 bytes in size.

### TECHNICAL FIELD

The present technology includes in vitro assays for identifying neutralizing antibodies (NAbs).

### BACKGROUND

Anti-Tumor Necrosis Factor-alpha (TNFα) antibodies are novel biologics that may potentially be used for the treatment of a variety of diseases. One hurdle to the development and implementation of anti-TNFα antibodies as a treatment is the potential for a subject administered such anti-TNFα antibodies to elicit an immune response against them. A subject's immunogenicity toward the anti-TNFα antibodies, particularly the subject's development of antibodies that neutralize the anti-TNFα antibody, would thereby reduce efficacy of the anti-TNFα antibody to treat the subject's disease. To advance the development of treatments that use anti-TNFα antibodies, there is need for assays that reliably determine the presence of anti-TNFα neutralizing antibodies and amounts thereof in subjects receiving or having been administered the anti-TNFα antibody.

### SUMMARY

In some embodiments, the present technology comprises methods for detecting a neutralizing antibody (NAb) against a target antibody in a sample comprising: (i) disassociating the NAb from the target antibody by adding an acid wash to the sample; (ii) purifying the NAb in the sample; (iii) contacting the purified NAb with a detection antibody comprising an anti-TNFα antibody conjugated to a detection label; (iv) contacting the purified NAb and the detection antibody with a TNFα-coated substrate; and (v) detecting the NAb when the detection antibody binds the NAb instead of the TNFα-coated substrate, wherein the target antibody comprises an anti-TNFα chimeric human-murine monoclonal IgG1-kappa antibody comprising SEQ ID NO: 9-11 or 15-17 and SEQ ID NOs: 12-14 or 18-20.

In some embodiments, the method further comprises detecting the detection label before detecting the NAb.

In some embodiments, the method further comprises washing the TNFα-coated substrate before detecting the NAb.

In some embodiments, the acid wash comprises acetic acid.

In some embodiments, purifying the NAb of step (ii) comprises (a) contacting the sample with an affinity antibody comprising anti-TNFα antibody conjugated to a first affinity label; (b) contacting the sample with a first solid phase coated with a second affinity label, wherein the first affinity label binds to the second affinity label; (c) washing the first solid phase; and (d) eluting the NAb from the first solid phase.

In some embodiments, the first affinity label is biotin.

In some embodiments, the first solid phase is a microbead. In some embodiments, the first solid phase is a magnetic microbead.

In some embodiments, the second affinity label is streptavidin.

In some embodiments, the NAb is purified using an automated purification system.

In some embodiments, the detection label includes a luminescent compound. In some embodiments, the present technology comprises a method, wherein the detection label includes an enzyme configured to convert a chromogenic substrate into a pigment. In some embodiments, the present technology comprises a method, wherein the detection label is a third affinity label.

In some embodiments, detecting the detection label further comprises contacting the third affinity label with a fourth affinity label conjugated to a luminescent compound or enzyme configured to convert a chromogenic substrate into a pigment, wherein the third affinity label binds to the fourth affinity label.

In some embodiments, the third affinity label is biotin, and the fourth affinity label is streptavidin.

In some embodiments, detecting the detection label comprises detecting the luminescent compound. In some embodiments, detecting the detection label comprises converting the chromogenic substrate into the pigment and detecting the pigment. In some embodiments, detecting the detection label comprises converting the chromogenic substrate into the pigment and detecting an optical density of the sample.

In some embodiments, the enzyme is a horseradish peroxidase (HRP).

In some embodiments, the chromogenic substrate is 3,3′,5,5′-tetramethylbenzidine (TMB).

In some embodiments, steps (i) to (v) are performed on a negative control sample having no detectable NAb.

In some embodiments, the detection antibody binding the NAb instead of the TNFα-coated substrate is detected when the detection label is present at a level below a predetermined level. In some embodiments, the detection label is present at the level below the predetermined level when an optical density of the sample is above a predetermined cut-point.

In some embodiments, the predetermined cut-point is the level corresponding to a false-positive rate of 3% or less. In some embodiments, the predetermined cut-point is the level corresponding to a 1% false-positive rate. In some embodiments, the predetermined cut-point is 0.5-2. In some embodiments, the predetermined cut-point is 0.91.

In some embodiments, a sensitivity of detecting the NAb is less than 500 ng/mL. In some embodiments, the sensitivity of detecting the NAb is less than 250 ng/mL. In some embodiments, the sensitivity of detecting the NAb is less than 150 ng/mL.

In some embodiments, a target tolerance is about 1000 ng/mL.

In some embodiments, the target antibody comprises SEQ ID NOs: 2 and 5. In some embodiments, the target antibody comprises SEQ ID NOs: 1 and 4.

In some embodiments, the present technology comprises a method for detecting a NAb (neutralizing antibody) against a target antibody in a sample comprising: (i) purifying the NAb in the sample; (ii) determining whether the NAb is present in the sample by performing a competitive binding assay, wherein the NAb is present if the NAb binds the anti-TNFα antibody with a higher affinity than the affinity of the anti-TNFα antibody to TNFα; and (iii) detecting the NAb; wherein a sensitivity of the method is 250 ng/mL or less, wherein the target antibody comprises an anti-TNFα chimeric human-murine monoclonal IgG1-kappa antibody comprising SEQ ID NO: 9-11 or 15-17 and SEQ ID NOs: 12-14 or 18-20.

In some embodiments, before the NAb is purified, the method further comprises disassociating the NAb from the target antibody by adding an acid wash to the sample.

In some embodiments, the competitive binding assay comprises: (a) contacting the purified NAb with a detection antibody comprising an anti-TNFα antibody conjugated to a detection label; (b) contacting the purified NAb and the detection antibody with a TNFα-coated substrate; (c) washing the TNFα-coated substrate; and (d) detecting the detection label.

In some embodiments, detecting the NAb comprises determining whether the detection label is present at a level below a predetermined level.

In some embodiments, the detection label is present at the level below the predetermined level when an optical density is above a predetermined cut-point.

In some embodiments, the acid wash comprises acetic acid.

In some embodiments, purifying the NAb comprises: (a) contacting the sample with an affinity antibody comprising an anti-TNFα antibody conjugated to a first affinity label; (b) contacting the sample with a first solid phase coated with a second affinity label, wherein the first affinity label binds to the second affinity label; (c) washing the first solid phase; and (d) eluting the NAb from the first solid phase.

In some embodiments, the first affinity label is biotin.

In some embodiments, the first solid phase is a microbead. In some embodiments, the first solid phase is a magnetic microbead.

In some embodiments, the second affinity label is streptavidin.

In some embodiments, the NAb is purified using an automated purification system.

In some embodiments, the detection label comprises a luminescent compound.

In some embodiments, the detection label comprises an enzyme configured to convert a chromogenic substrate into a pigment. In some embodiments, the detection label is a third affinity label.

In some embodiments, detecting the detection label further comprises contacting the third affinity label with a fourth affinity label conjugated to a luminescent compound or enzyme configured to convert a chromogenic substrate into a pigment, wherein the third affinity label binds to the fourth affinity label.

In some embodiments, the third affinity label is biotin, and the fourth affinity label is streptavidin.

In some embodiments, detecting the detection label comprises detecting the luminescent compound. In some embodiments, detecting the detection label comprises converting the chromogenic substrate into the pigment and detecting the pigment. In some embodiments, detecting the detection label comprises converting the chromogenic substrate into the pigment and detecting an optical density of the sample.

In some embodiments, the enzyme is a horseradish peroxidase (HRP).

In some embodiments, the chromogenic substrate is 3,3′,5,5′-tetramethylbenzidine (TMB).

In some embodiments, steps (i) to (iii) are performed on a negative control sample having no detectable NAb.

In some embodiments, the predetermined cut-point is a level corresponding to a 1% false-positive rate. In some embodiments, the predetermined cut-point is 0.91.

In some embodiments, the sensitivity of detecting the NAb is less than 500 ng/mL. In some embodiments, the sensitivity of detecting the NAb is less than 250 ng/mL. In some embodiments, the sensitivity of detecting the NAb is less than 150 ng/mL.

In some embodiments, a target tolerance is about 1000 ng/mL.

In some embodiments, the target antibody comprises SEQ ID NOs: 2 and 5. In some embodiments, the target antibody comprises SEQ ID NOs: 1 and 4.

In some embodiments, the present technology comprises a kit for detecting a neutralizing antibody (NAb) against a target antibody comprising an anti-TNFα chimeric human-murine monoclonal IgG1-kappa antibody comprising SEQ ID NO: 9-11 or 15-17 and SEQ ID NOs: 12-14 or 18-20 in a sample comprising: (i) an affinity antibody comprising the target antibody conjugated to a first affinity label; (ii) a first solid phase coated with a second affinity label; (iii) a detection antibody comprising the target antibody conjugated to a detection label; (iv) a substrate; and (v) TNFα, wherein the target antibody comprises an anti-TNFα chimeric human-murine monoclonal IgG1-kappa antibody comprising SEQ ID NO: 9-11 or 15-17 and SEQ ID NOs: 12-14 or 18-20.

In some embodiments, the first affinity label is biotin.

In some embodiments, the second affinity label is streptavidin.

In some embodiments, the detection label is a luminescent compound, an enzyme configured to convert a chromogenic substrate into a pigment, or biotin.

In some embodiments, the first solid phase is a microbead. In some embodiments, the first solid phase is a magnetic microbead.

In some embodiments, the detection label is biotin.

In some embodiments, the kit further comprising an acid wash buffer.

In some embodiments, the kit further comprising a washing buffer.

In some embodiments, the kit further comprising an elution buffer.

### BRIEF DESCRIPTION OF THE DRAWINGS

FIG. 1 shows a flow diagram of an example method of the present technology.

FIG. 2 shows a flow diagram of another example method of the present technology.

FIG. 3 shows an example normal distribution of 295 log-transformed normalized values from control samples prepared according to the methods of the present technology.

### DETAILED DESCRIPTION

The present technology comprises methods for detecting a neutralizing antibody (NAb) against a target antibody. The target antibody may comprise an anti-TNFα antibody. In some embodiments, the anti-TNFα antibody is a chimeric human-murine monoclonal IgG1-kappa antibody. In some embodiments, the target antibody comprises heavy chain complementarity-determining regions (CDRs) of SEQ ID NOs: 9-11 and light chain CDRs of SEQ ID NOs: 12-14. In some embodiments, the target antibody comprises heavy chain CDRs of SEQ ID NOs: 15-17 and light chain CDRs of SEQ ID NOs: 18-20. In some embodiments, the target antibody comprises a heavy chain variable region of SEQ ID NO: 2 and a light chain variable region of SEQ ID NO: 5. In some embodiments, the target antibody comprises a heavy chain of SEQ ID NOs: 1 and light chain of SEQ ID NO: 4. in a sample obtained from a subject receiving or having been administered the target antibody. The present technology also comprises a kit for detecting a NAb in the sample according to the methods of the present technology.

### Definitions

The term “about” means a quantity, level, value, number, frequency, percentage, dimension, size, amount, weight or length that varies by acceptable levels in the art. Typically, such variation may be as much 10% above and below a reference quantity, level, value, number, frequency, percentage, dimension, size, amount, weight or length and such variation may be influenced by standard applicable measurement practices. When the term “about” is used in conjunction with a numerical range, it modifies that range by extending the boundaries above and below the numerical values set forth.

As used herein, an “affinity antibody” comprises an anti-TNFα antibody. In some embodiments, the affinity antibody comprises the target antibody. The affinity antibody may also comprise an affinity label that has a high affinity with a separate label. For example, the affinity label may be a first affinity label (i.e., biotin) that has a high affinity with a second affinity label (i.e., streptavidin). In some embodiments, the affinity antibody comprises an anti-TNFα antibody and an affinity label. In some embodiments, the affinity antibody comprises the target antibody and an affinity label.

As used herein, a “detection antibody” comprises a TNFα antibody. In some embodiments, the detection antibody comprises the target antibody. The detection antibody may also comprise a detection label that may be directly or indirectly detected. In some embodiments, the detection antibody comprises an anti-TNFα antibody and a detection label. In some embodiments, the detection antibody comprises the target antibody and a detection label.

In some embodiments, the method for detecting an NAb against the target antibody in the sample comprises (i) disassociating the NAb from the target antibody by adding an acid wash to the sample; (ii) purifying the NAb from the sample; (iii) contacting the purified NAb with an anti-TNFα antibody conjugate comprising the anti-TNFα antibody conjugated to a detection label (i.e., a “detection antibody”); (iv) contacting the purified NAb and the detection antibody with a TNFα-coated substrate; and (v) detecting the NAb by determining whether the detection label is present at a level above a predetermined cut-point.

In some embodiments, the method for detecting an NAb against the target antibody comprises (i) purifying the NAb in the sample; (ii) determining whether the NAb is present in the sample by performing a competitive binding assay, wherein the NAb is present if it binds the detection antibody with a higher affinity than a binding affinity of the detection antibody to TNFα; and (iii) detecting the NAb; wherein a sensitivity of the method is about 250 ng/mL or less.

In some embodiments, the competitive binding assay comprises (a) contacting the purified NAb with the detection antibody; (b) contacting the purified NAb and the detection antibody with the TNFα-coated substrate; (c) washing the TNFα-coated substrate; and (d) detecting the detection label.

The sample may be a sample obtained from a mammal, including human, rat, mouse, rabbit, or other animal. In some embodiments, the sample is a blood, plasma, or serum sample. In some embodiments, the sample is plasma. In some embodiments, the sample is serum. In some embodiments, the sample is pre-processed before the NAb is detected according to the methods of the present technology. As used herein, “pre-processing” includes extracting tissue from a subject, and identifying the sample (e.g., drawing a blood sample and identifying or isolating a plasma or serum sample).

### Antibodies

### Neutralizing Antibodies (NAbs)

During or after administration of a drug (e.g., a therapeutic drug) to a subject, the subject's immune system may respond by generating NAbs. NAbs may bind at least a portion of the drug, thereby sequestering, neutralizing, and/or reducing efficacy of the drug. In some embodiments, the drug is a drug antibody. When NAbs are present, the NAb may bind at least a portion of the drug antibody, including, but not limited to, binding to one or more amino acids of a heavy chain, a light chain, a variable region (e.g., a heavy chain variable region domain (V_{H}) or a light chain variable region domain (V_{L})), a constant region, an Fc domain, an Fab domain, a hinge region, a disulfide bond, or a complementarity-determining region (CDR) of the drug antibody.

NAbs may be naturally produced as part of a subject's innate immune response. The NAb may be a natural antibody produced by the subject, for example by lymphocytes using variable (diversity) joining (V(D)J) recombination. For example, the NAb may be an immunoglobulin. In some embodiments, the NAb is an IgG, IgA, IgE, monoclonal antibody, polyclonal antibody, or any other kind of antibody that is capable of binding to the target antibody.

### Target Antibodies

Presence of NAbs and/or binding of NAbs to the drug antibodies may be detected using antibodies which are the same as or mimic the drug antibodies (i.e., “target antibodies”). In some embodiments, the target antibodies comprise one or more anti-TNFα antibodies. The anti-TNFα antibodies are antibodies which bind at least a portion of a TNFα protein or peptide.

In some embodiments, the target antibodies comprise a monoclonal antibody.

In some embodiments, the target antibodies of the present technology are chimeric antibodies. Nonlimiting examples of chimeric antibodies include human-murine antibodies, human-rat antibodies, human-hamster antibodies, human-rabbit antibodies, human-bovine antibodies, and human-goat antibodies. In some embodiments, the chimeric antibodies comprise a humanized antibody (e.g., a full humanized antibody or a partially humanized antibody).

In some embodiments, the target antibodies of the present technology may comprise an isotype that influences functional properties and/or structural features of the antibody. In some embodiments the target antibodies comprise an isotype selected from the group consisting of IgG (e.g., IgG1, IgG2, IgG3, IgG4), IgM, IgA, IgD, and IgE. The light chain may comprise isotype kappa (κ) or isotype lambda (λ). In some embodiments, the target antibody comprises an IgG1-kappa antibody.

### Target Antibody Sequences

The target antibodies of the present technology may comprise an anti-TNFα antibody or at least a portion of the anti-TNFα antibody. For example, the target antibodies may comprise an amino acid sequence derived from one or more of a heavy chain, a light chain, a variable region (e.g., V_{H} or V_{L} domains), a constant region, an Fc domain, a Fab domain, a hinge region, a disulfide bond, or a CDR of an anti-TNFα antibody. The NAbs of the present technology may bind to one or more amino acid residues of the amino acid sequence.

In some embodiments, the portion of the anti-TNFα antibody comprises a sequence that is about 75%, 80%, 85%, 90%, 95%, 99%, or 100% identical to an amino acid sequence in Table 1.

In some embodiments, the portion of the anti-TNFα antibody comprises a sequence that is at least 75%, 80%, 85%, 90%, 95%, 99%, or 100% identical to an amino acid sequence in Table 1.

In some embodiments, the portion of the anti-TNFα antibody comprises a sequence that is at least about 75%, 80%, 85%, 90%, 95%, 99%, or 100% identical to an amino acid sequence in Table 1.

### Heavy Chain:

The target antibodies of the present technology comprise a heavy chain having an amino acid sequence that is about 75%, 80%, 85%, 90%, 95%, 99%, or 100% identical to SEQ. ID NO: 1.

The target antibodies of the present technology comprise a heavy chain having an amino acid sequence that is at least 75%, 80%, 85%, 90%, 95%, 99%, or 100% identical to SEQ. ID NO: 1.

The target antibodies of the present technology comprise a heavy chain having an amino acid sequence that is at least about 75%, 80%, 85%, 90%, 95%, 99%, or 100% identical to SEQ. ID NO: 1.

The heavy chain of the target antibodies may comprise a variable region domain (V_{H}) having an amino acid sequence that is about 75%, 80%, 85%, 90%, 95%, 99%, or 100% identical to SEQ. ID NO: 2.

The heavy chain of the target antibodies may comprise a V_{H} having an amino acid sequence that is at least 75%, 80%, 85%, 90%, 95%, 99%, or 100% identical to SEQ. ID NO: 2.

The heavy chain of the target antibodies may comprise a V_{H} having an amino acid sequence that is at least about 75%, 80%, 85%, 90%, 95%, 99%, or 100% identical to SEQ. ID NO: 2.

The heavy chain of the target antibodies may comprise a constant region having an amino acid sequence that is about 75%, 80%, 85%, 90%, 95%, 99%, or 100% identical to SEQ. ID NO: 3.

The heavy chain of the target antibodies may comprise a constant region having an amino acid sequence that is at least 75%, 80%, 85%, 90%, 95%, 99%, or 100% identical to SEQ. ID NO: 3.

The heavy chain of the target antibodies may comprise a constant region having an amino acid sequence that is at least about 75%, 80%, 85%, 90%, 95%, 99%, or 100% identical to SEQ. ID NO: 3.

The target antibodies of the present technology comprise a heavy chain having at least one Complementarity Determining Region (CDR) sequence. The at least one CDR sequence confers binding to a specific antigen (e.g., a TNFα peptide).

In some embodiments, the target antibodies comprise a heavy chain having a CDR having an amino acid sequence that is about 75%, 80%, 85%, 90%, 95%, 99%, or 100% identical to SEQ. ID NO: 9.

In some embodiments, the target antibodies comprise a heavy chain having a CDR having an amino acid sequence that is at least 75%, 80%, 85%, 90%, 95%, 99%, or 100% identical to SEQ. ID NO: 9.

In some embodiments, the target antibodies comprise a heavy chain having a CDR having an amino acid sequence that is at least about 75%, 80%, 85%, 90%, 95%, 99%, or 100% identical to SEQ. ID NO: 9.

In some embodiments, the target antibodies comprise a heavy chain having a CDR having an amino acid sequence that is about 75%, 80%, 85%, 90%, 95%, 99%, or 100% identical to SEQ. ID NO: 10.

In some embodiments, the target antibodies comprise a heavy chain having a CDR having an amino acid sequence that is at least 75%, 80%, 85%, 90%, 95%, 99%, or 100% identical to SEQ. ID NO: 10.

In some embodiments, the target antibodies comprise a heavy chain having a CDR having an amino acid sequence that is at least about 75%, 80%, 85%, 90%, 95%, 99%, or 100% identical to SEQ. ID NO: 10.

In some embodiments, the target antibodies comprise a heavy chain having a CDR having an amino acid sequence that is about 75%, 80%, 85%, 90%, 95%, 99%, or 100% identical to SEQ. ID NO: 11.

In some embodiments, the target antibodies comprise a heavy chain having a CDR having an amino acid sequence that is at least 75%, 80%, 85%, 90%, 95%, 99%, or 100% identical to SEQ. ID NO: 11.

In some embodiments, the target antibodies comprise a heavy chain having a CDR having an amino acid sequence that is at least about 75%, 80%, 85%, 90%, 95%, 99%, or 100% identical to SEQ. ID NO: 11.

In some embodiments, the target antibodies comprise a heavy chain having a CDR having an amino acid sequence that is about 75%, 80%, 85%, 90%, 95%, 99%, or 100% identical to SEQ. ID NO: 15.

In some embodiments, the target antibodies comprise a heavy chain having a CDR having an amino acid sequence that is at least 75%, 80%, 85%, 90%, 95%, 99%, or 100% identical to SEQ. ID NO: 15.

In some embodiments, the target antibodies comprise a heavy chain having a CDR having an amino acid sequence that is at least about 75%, 80%, 85%, 90%, 95%, 99%, or 100% identical to SEQ. ID NO: 15.

In some embodiments, the target antibodies comprise a heavy chain having a CDR having an amino acid sequence that is about 75%, 80%, 85%, 90%, 95%, 99%, or 100% identical to SEQ. ID NO: 16.

In some embodiments, the target antibodies comprise a heavy chain having a CDR having an amino acid sequence that is at least 75%, 80%, 85%, 90%, 95%, 99%, or 100% identical to SEQ. ID NO: 16.

In some embodiments, the target antibodies comprise a heavy chain having a CDR having an amino acid sequence that is at least about 75%, 80%, 85%, 90%, 95%, 99%, or 100% identical to SEQ. ID NO: 16.

In some embodiments, the target antibodies comprise a heavy chain having a CDR having an amino acid sequence that is about 75%, 80%, 85%, 90%, 95%, 99%, or 100% identical to SEQ. ID NO: 17.

In some embodiments, the target antibodies comprise a heavy chain having a CDR having an amino acid sequence that is at least 75%, 80%, 85%, 90%, 95%, 99%, or 100% identical to SEQ. ID NO: 17.

In some embodiments, the target antibodies comprise a heavy chain having a CDR having an amino acid sequence that is at least about 75%, 80%, 85%, 90%, 95%, 99%, or 100% identical to SEQ. ID NO: 17.

In some embodiments, the target antibodies comprise a heavy chain comprising (1) a CDR having an amino acid sequence that is about 75%, 80%, 85%, 90%, 95%, 99%, or 100% identical to SEQ. ID NO: 9, (2) a CDR having an amino acid sequence that is about 75%, 80%, 85%, 90%, 95%, 99%, or 100% identical to SEQ. ID NO: 10, and (3) a CDR having an amino acid sequence that is about 75%, 80%, 85%, 90%, 95%, 99%, or 100% identical to SEQ. ID NO: 11.

In some embodiments, the target antibodies comprise a heavy chain comprising (1) a CDR having an amino acid sequence that is at least 75%, 80%, 85%, 90%, 95%, 99%, or 100% identical to SEQ. ID NO: 9, (2) a CDR having an amino acid sequence that is at least 75%, 80%, 85%, 90%, 95%, 99%, or 100% identical to SEQ. ID NO: 10, and (3) a CDR having an amino acid sequence that is at least 75%, 80%, 85%, 90%, 95%, 99%, or 100% identical to SEQ. ID NO: 11.

In some embodiments, the target antibodies comprise a heavy chain comprising (1) a CDR having an amino acid sequence that is at least about 75%, 80%, 85%, 90%, 95%, 99%, or 100% identical to SEQ. ID NO: 9, (2) a CDR having an amino acid sequence that is at least about 75%, 80%, 85%, 90%, 95%, 99%, or 100% identical to SEQ. ID NO: 10, and (3) a CDR having an amino acid sequence that is at least about 75%, 80%, 85%, 90%, 95%, 99%, or 100% identical to SEQ. ID NO: 11.

In some embodiments, the target antibodies comprise a heavy chain comprising (1) a CDR having an amino acid sequence that is about 75%, 80%, 85%, 90%, 95%, 99%, or 100% identical to SEQ. ID NO: 15, (2) a CDR having an amino acid sequence that is about 75%, 80%, 85%, 90%, 95%, 99%, or 100% identical to SEQ. ID NO: 16, and (3) a CDR having an amino acid sequence that is about 75%, 80%, 85%, 90%, 95%, 99%, or 100% identical to SEQ. ID NO: 17.

In some embodiments, the target antibodies comprise a heavy chain comprising (1) a CDR having an amino acid sequence that is at least 75%, 80%, 85%, 90%, 95%, 99%, or 100% identical to SEQ. ID NO: 15, (2) a CDR having an amino acid sequence that is at least 75%, 80%, 85%, 90%, 95%, 99%, or 100% identical to SEQ. ID NO: 16, and (3) a CDR having an amino acid sequence that is at least 75%, 80%, 85%, 90%, 95%, 99%, or 100% identical to SEQ. ID NO: 17.

In some embodiments, the target antibodies comprise a heavy chain comprising (1) a CDR having an amino acid sequence that is at least about 75%, 80%, 85%, 90%, 95%, 99%, or 100% identical to SEQ. ID NO: 15, (2) a CDR having an amino acid sequence that is at least about 75%, 80%, 85%, 90%, 95%, 99%, or 100% identical to SEQ. ID NO: 16, and (3) a CDR having an amino acid sequence that is at least about 75%, 80%, 85%, 90%, 95%, 99%, or 100% identical to SEQ. ID NO: 17.

In some embodiments, the heavy chain of the target antibodies of the present technology is encoded by a nucleotide sequence that is about 75%, 80%, 85%, 90%, 95%, 99%, or 100% identical to SEQ. ID NO: 8.

In some embodiments, the heavy chain of the target antibodies of the present technology is encoded by a nucleotide sequence that is at least 75%, 80%, 85%, 90%, 95%, 99%, or 100% identical to SEQ. ID NO: 8.

In some embodiments, the heavy chain of the target antibodies of the present technology is encoded by a nucleotide sequence that is at least about 75%, 80%, 85%, 90%, 95%, 99%, or 100% identical to SEQ. ID NO: 8.

### Light Chain:

The target antibodies of the present technology comprise a light chain having an amino acid sequence that is about 75%, 80%, 85%, 90%, 95%, 99%, or 100% identical to SEQ. ID NO: 4.

The target antibodies of the present technology comprise a light chain having an amino acid sequence that is at least 75%, 80%, 85%, 90%, 95%, 99%, or 100% identical to SEQ. ID NO: 4.

The target antibodies of the present technology comprise a light chain having an amino acid sequence that is at least about 75%, 80%, 85%, 90%, 95%, 99%, or 100% identical to SEQ. ID NO: 4.

The light chain of the target antibodies may comprise a variable region domain (V_{L}) having an amino acid sequence that is about 75%, 80%, 85%, 90%, 95%, 99%, or 100% identical to SEQ. ID NO: 5.

The light chain of the target antibodies may comprise a V_{L} having an amino acid sequence that is at least 75%, 80%, 85%, 90%, 95%, 99%, or 100% identical to SEQ. ID NO: 5.

The light chain of the target antibodies may comprise a V_{L} having an amino acid sequence that is at least about 75%, 80%, 85%, 90%, 95%, 99%, or 100% identical to SEQ. ID NO: 5.

The light chain of the target antibodies may comprise a constant region having an amino acid sequence that is about 75%, 80%, 85%, 90%, 95%, 99%, or 100% identical to SEQ. ID NO: 6.

The light chain of the target antibodies may comprise a constant region having an amino acid sequence that is at least 75%, 80%, 85%, 90%, 95%, 99%, or 100% identical to SEQ. ID NO: 6.

The light chain of the target antibodies may comprise a constant region having an amino acid sequence that is at least about 75%, 80%, 85%, 90%, 95%, 99%, or 100% identical to SEQ. ID NO: 6.

The target antibodies of the present technology comprise a light chain having at least one CDR sequence. The at least one CDR sequence confers binding to a specific antigen (e.g., a TNFα peptide).

In some embodiments, the target antibodies comprise a light chain having a CDR having an amino acid sequence that is about 75%, 80%, 85%, 90%, 95%, 99%, or 100% identical to SEQ. ID NO: 12.

In some embodiments, the target antibodies comprise a light chain having a CDR having an amino acid sequence that is at least 75%, 80%, 85%, 90%, 95%, 99%, or 100% identical to SEQ. ID NO: 12.

In some embodiments, the target antibodies comprise a light chain having a CDR having an amino acid sequence that is at least about 75%, 80%, 85%, 90%, 95%, 99%, or 100% identical to SEQ. ID NO: 12.

In some embodiments, the target antibodies comprise a light chain having a CDR having an amino acid sequence that is about 75%, 80%, 85%, 90%, 95%, 99%, or 100% identical to SEQ. ID NO: 13.

In some embodiments, the target antibodies comprise a light chain having a CDR having an amino acid sequence that is at least 75%, 80%, 85%, 90%, 95%, 99%, or 100% identical to SEQ. ID NO: 13.

In some embodiments, the target antibodies comprise a light chain having a CDR having an amino acid sequence that is at least about 75%, 80%, 85%, 90%, 95%, 99%, or 100% identical to SEQ. ID NO: 13.

In some embodiments, the target antibodies comprise a light chain having a CDR having an amino acid sequence that is about 75%, 80%, 85%, 90%, 95%, 99%, or 100% identical to SEQ. ID NO: 14.

In some embodiments, the target antibodies comprise a light chain having a CDR having an amino acid sequence that is at least 75%, 80%, 85%, 90%, 95%, 99%, or 100% identical to SEQ. ID NO: 14.

In some embodiments, the target antibodies comprise a light chain having a CDR having an amino acid sequence that is at least about 75%, 80%, 85%, 90%, 95%, 99%, or 100% identical to SEQ. ID NO: 14.

In some embodiments, the target antibodies comprise a light chain having a CDR having an amino acid sequence that is about 75%, 80%, 85%, 90%, 95%, 99%, or 100% identical to SEQ. ID NO: 18.

In some embodiments, the target antibodies comprise a light chain having a CDR having an amino acid sequence that is at least 75%, 80%, 85%, 90%, 95%, 99%, or 100% identical to SEQ. ID NO: 18.

In some embodiments, the target antibodies comprise a light chain having a CDR having an amino acid sequence that is at least about 75%, 80%, 85%, 90%, 95%, 99%, or 100% identical to SEQ. ID NO: 18.

In some embodiments, the target antibodies comprise a light chain having a CDR having an amino acid sequence that is about 75%, 80%, 85%, 90%, 95%, 99%, or 100% identical to SEQ. ID NO: 19.

In some embodiments, the target antibodies comprise a light chain having a CDR having an amino acid sequence that is at least 75%, 80%, 85%, 90%, 95%, 99%, or 100% identical to SEQ. ID NO: 19.

In some embodiments, the target antibodies comprise a light chain having a CDR having an amino acid sequence that is at least about 75%, 80%, 85%, 90%, 95%, 99%, or 100% identical to SEQ. ID NO: 19.

In some embodiments, the target antibodies comprise a light chain having a CDR having an amino acid sequence that is about 75%, 80%, 85%, 90%, 95%, 99%, or 100% identical to SEQ. ID NO: 20.

In some embodiments, the target antibodies comprise a light chain having a CDR having an amino acid sequence that is at least 75%, 80%, 85%, 90%, 95%, 99%, or 100% identical to SEQ. ID NO: 20.

In some embodiments, the target antibodies comprise a light chain having a CDR having an amino acid sequence that is at least about 75%, 80%, 85%, 90%, 95%, 99%, or 100% identical to SEQ. ID NO: 20.

In some embodiments, the target antibodies comprise a light chain comprising (1) a CDR having an amino acid sequence that is about 75%, 80%, 85%, 90%, 95%, 99%, or 100% identical to SEQ. ID NO: 12, (2) a CDR having an amino acid sequence that is about 75%, 80%, 85%, 90%, 95%, 99%, or 100% identical to SEQ. ID NO: 13, and (3) a CDR having an amino acid sequence that is about 75%, 80%, 85%, 90%, 95%, 99%, or 100% identical to SEQ. ID NO: 14.

In some embodiments, the target antibodies comprise a light chain comprising (1) a CDR having an amino acid sequence that is at least 75%, 80%, 85%, 90%, 95%, 99%, or 100% identical to SEQ. ID NO: 12, (2) a CDR having an amino acid sequence that is at least 75%, 80%, 85%, 90%, 95%, 99%, or 100% identical to SEQ. ID NO: 13, and (3) a CDR having an amino acid sequence that is at least 75%, 80%, 85%, 90%, 95%, 99%, or 100% identical to SEQ. ID NO: 14.

In some embodiments, the target antibodies comprise a light chain comprising (1) a CDR having an amino acid sequence that is at least about 75%, 80%, 85%, 90%, 95%, 99%, or 100% identical to SEQ. ID NO: 12, (2) a CDR having an amino acid sequence that is at least about 75%, 80%, 85%, 90%, 95%, 99%, or 100% identical to SEQ. ID NO: 13, and (3) a CDR having an amino acid sequence that is at least about 75%, 80%, 85%, 90%, 95%, 99%, or 100% identical to SEQ. ID NO: 14.

In some embodiments, the target antibodies comprise a light chain comprising (1) a CDR having an amino acid sequence that is about 75%, 80%, 85%, 90%, 95%, 99%, or 100% identical to SEQ. ID NO: 18, (2) a CDR having an amino acid sequence that is about 75%, 80%, 85%, 90%, 95%, 99%, or 100% identical to SEQ. ID NO: 19, and (3) a CDR having an amino acid sequence that is about 75%, 80%, 85%, 90%, 95%, 99%, or 100% identical to SEQ. ID NO: 20.

In some embodiments, the target antibodies comprise a light chain comprising (1) a CDR having an amino acid sequence that is at least 75%, 80%, 85%, 90%, 95%, 99%, or 100% identical to SEQ. ID NO: 18, (2) a CDR having an amino acid sequence that is at least 75%, 80%, 85%, 90%, 95%, 99%, or 100% identical to SEQ. ID NO: 19, and (3) a CDR having an amino acid sequence that is at least 75%, 80%, 85%, 90%, 95%, 99%, or 100% identical to SEQ. ID NO: 20.

In some embodiments, the target antibodies comprise a light chain comprising (1) a CDR having an amino acid sequence that is at least about 75%, 80%, 85%, 90%, 95%, 99%, or 100% identical to SEQ. ID NO: 18, (2) a CDR having an amino acid sequence that is at least about 75%, 80%, 85%, 90%, 95%, 99%, or 100% identical to SEQ. ID NO: 19, and (3) a CDR having an amino acid sequence that is at least about 75%, 80%, 85%, 90%, 95%, 99%, or 100% identical to SEQ. ID NO: 20.

In some embodiments, the light chain of the target antibodies of the present technology is encoded by a nucleotide sequence that is about 75%, 80%, 85%, 90%, 95%, 99%, or 100% identical to SEQ. ID NO: 7.

In some embodiments, the light chain of the target antibodies of the present technology is encoded by a nucleotide sequence that is at least 75%, 80%, 85%, 90%, 95%, 99%, or 100% identical to SEQ. ID NO: 7.

In some embodiments, the light chain of the target antibodies of the present technology is encoded by a nucleotide sequence that is at least about 75%, 80%, 85%, 90%, 95%, 99%, or 100% identical to SEQ. ID NO: 7.

TABLE 1
Sequences of Example Anti-TNFα Antibodies of the Present Technology

| SEQ ID | | |
|---|---|---|
| NO: | Description | Sequence |
| 1 | aa sequence of | EVKLEESGGGLVQPGGSMKLSCVASGFIFSNHWMNWVRQ |
| | Heavy Chain of | SPEKGLEWVAEIRSKSINSATHYAESVKGRFTISRDDSKSAV |
| | anti-TNFα | YLQMTDLRTEDTGVYYCSRNYYGSTYDYWGQGTTLTVSS |
| | antibody | ASTKGPSVFPLAPSSKSTSGGTAALGCLVKDYFPEPVTVSW |
| | (XTMAB-16, | NSGALTSGVHTFPAVLQSSGLYSLSSVVTVPSSSLGTQTYIC |
| | target antibody) | NVNHKPSNTKVDKKVEPKSCDKTHTCPPCPAPELLGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALPAPIEKTISKAKGQPREPQVYTLPPSRDELTKNQ |
| | | VSLTCLVKGFYPSDIAVEWESNGQPENNYKTTPPVLDSDGS |
| | | FFLYSKLTVDKSRWQQGNVFSCSVMHEALHNHYTQKSLSL |
| | | SPGK |
| 2 | aa sequence of | EVKLEESGGGLVQPGGSMKLSCVASGFIFSNHWMNWVRQ |
| | Heavy chain | SPEKGLEWVAEIRSKSINSATHYAESVKGRFTISRDDSKSAV |
| | variable region | YLQMTDLRTEDTGVYYCSRNYYGSTYDYWGQGTTLTV |
| | of anti-TNFα | |
| | antibody | |
| | (XTMAB-16, | |
| | target antibody) | |
| 3 | aa sequence of | SSASTKGPSVFPLAPSSKSTSGGTAALGCLVKDYFPEPVTVS |
| | Heavy chain | WNSGALTSGVHTFPAVLQSSGLYSLSSVVTVPSSSLGTQTYI |
| | constant region | CNVNHKPSNTKVDKKVEPKSCDKTHTCPPCPAPELLGGPS |
| | of anti-TNFα | VFLFPPKPKDTLMISRTPEVTCVVVDVSHEDPEVKFNWYV |
| | antibody | DGVEVHNAKTKPREEQYNSTYRVVSVLTVLHQDWLNGKE |
| | (XTMAB-16, | YKCKVSNKALPAPIEKTISKAKGQPREPQVYTLPPSRDELTK |
| | target antibody) | NQVSLTCLVKGFYPSDIAVEWESNGQPENNYKTTPPVLDSD |
| | | GSFFLYSKLTVDKSRWQQGNVFSCSVMHEALHNHYTQKSL |
| | | SLSPGK |
| 4 | aa sequence of | DILLTQSPAILSVSPGERVSFSCRASQFVGSSIHWYQQRTNG |
| | Light chain of | SPRLLIKYASESMSGIPSRFSGSGSGTDFTLSINTVESEDIAD |
| | anti-TNFα | YYCQQSHSWPFTFGSGTNLEVKRTVAAPSVFIFPPSDEQLK |
| | antibody | SGTASVVCLLNNFYPREAKVQWKVDNALQSGNSQESVTE |
| | (XTMAB-16, | QDSKDSTYSLSSTLTLSKADYEKHKVYACEVTHQGLSSPVT |
| | target antibody) | KSFNRGEC |
| 5 | aa sequence of | DILLTQSPAILSVSPGERVSFSCRASQFVGSSIHWYQQRTNG |
| | Light chain | SPRLLIKYASESMSGIPSRFSGSGSGTDFTLSINTVESEDIAD |
| | variable region | YYCQQSHSWPFTFGSGTNLEV |
| | of anti-TNFα | |
| | antibody | |
| | (XTMAB-16, | |
| | target antibody) | |
| 6 | aa sequence of | KRTVAAPSVFIFPPSDEQLKSGTASVVCLLNNFYPREAKVQ |
| | Light chain | WKVDNALQSGNSQESVTEQDSKDSTYSLSSTLTLSKADYE |
| | constant region | KHKVYACEVTHQGLSSPVTKSFNRGEC |
| | of anti-TNFα | |
| | antibody | |
| | (XTMAB-16, | |
| | target antibody) | |
| 7 | Nucleotide | GCTAGCGAATTCCACCATGGGCTGGTCCTGCATCATCCT |
| | sequence | GTTTCTGGTGGCCACCGCCACCGGCGTGCACTCCGACAT |
| | encoding light | CCTGCTGACCCAGTCCCCCGCCATCCTGTCCGTGTCTCCC |
| | chain of anti- | GGCGAGCGGGTGTCCTTCTCCTGCCGGGCCTCCCAGTTC |
| | TNFα antibody | GTGGGCTCCTCCATCCACTGGTATCAGCAGCGGACCAAC |
| | (XTMAB-16, | GGCTCCCCTCGGCTGCTGATCAAGTACGCCTCCGAGTCC |
| | target antibody) | ATGTCCGGCATCCCTTCCCGGTTCTCCGGCTCCGGCTCTG |
| | | GCACCGACTTCACCCTGTCCATCAACACCGTGGAGTCCG |
| | | AGGATATTGCCGACTACTACTGCCAGCAGTCCCACTCCT |
| | | GGCCTTTCACCTTCGGCTCCGGCACCAACCTGGAGGTGA |
| | | AGCGTACGGTGGCTGCACCATCTGTCTTCATCTTCCCGCC |
| | | ATCTGATGAGCAGTTGAAATCTGGAACTGCCTCTGTTGT |
| | | GTGCCTGCTGAATAACTTCTATCCCAGAGAGGCCAAAGT |
| | | ACAGTGGAAGGTGGATAACGCCCTCCAATCGGGTAACTC |
| | | CCAGGAGAGTGTCACAGAGCAGGACAGCAAGGACAGCA |
| | | CCTACAGCCTCAGCAGCACCCTGACGCTGAGCAAAGCAG |
| | | ACTACGAGAAACACAAAGTCTACGCCTGCGAAGTCACCC |
| | | ATCAGGGCCTGAGCTCGCCCGTCACAAAGAGCTTCAACA |
| | | GGGGAGAGTGTTAA |
| 8 | Nucleotide | GCTAGCGATATCGAATTCTTTTCTTTTGTTTTGTTTCCACC |
| | sequence | ATGGGCTGGTCCTGCATCATCCTGTTTCTGGTGGCCACCG |
| | encoding heavy | CCACCGGCGTGCACTCCGAGGTGAAGCTGGAGGAGTCTG |
| | chain of anti- | GCGGCGGACTGGTGCAGCCTGGCGGCTCCATGAAGCTGT |
| | TNFα antibody | CCTGCGTGGCCTCCGGCTTCATCTTCTCCAACCACTGGAT |
| | (XTMAB-16, | GAACTGGGTGCGCCAGTCCCCTGAGAAGGGCCTGGAGT |
| | target antibody) | GGGTGGCCGAGATCCGGTCCAAGTCCATCAACTCCGCCA |
| | | CCCACTACGCCGAGTCCGTGAAGGGCCGGTTCACCATCT |
| | | CCCGGGACGACTCCAAGTCCGCCGTGTACCTGCAGATGA |
| | | CCGACCTGCGGACCGAGGACACCGGAGTGTACTACTGCT |
| | | CCCGGAACTACTACGGCTCCACCTACGACTACTGGGGCC |
| | | AGGGCACCACACTGACCGTGAGCTCCGCCTCCACCAAGG |
| | | GCCCATCGGTCTTCCCCCTGGCACCCTCCTCCAAGAGCA |
| | | CCTCTGGGGGCACAGCGGCCCTGGGCTGCCTGGTCAAGG |
| | | ACTACTTCCCCGAACCGGTGACGGTGTCGTGGAACTCAG |
| | | GCGCCCTGACCAGCGGCGTGCACACCTTCCCGGCTGTCC |
| | | TACAGTCCTCAGGACTCTACTCCCTCAGCAGCGTGGTGA |
| | | CCGTGCCCTCCAGCAGCTTGGGCACCCAGACCTACATCT |
| | | GCAACGTGAATCACAAGCCCAGCAACACCAAGGTGGAC |
| | | AAGAAAGTCGAGCCCAAATCTTGTGACAAAACTCACAC |
| | | ATGCCCACCGTGCCCAGCACCTGAACTCCTGGGGGGACC |
| | | GTCAGTCTTCCTCTTCCCCCCAAAACCCAAGGACACCCT |
| | | CATGATCTCCCGGACCCCTGAGGTCACATGCGTGGTGGT |
| | | GGACGTGAGCCACGAAGACCCTGAGGTCAAGTTCAACT |
| | | GGTACGTGGACGGCGTGGAGGTGCATAATGCCAAGACA |
| | | AAGCCGCGGGAGGAGCAGTACAACAGCACGTACCGTGT |
| | | GGTCAGCGTCCTCACCGTCCTGCACCAGGACTGGCTGAA |
| | | TGGCAAGGAGTACAAGTGCAAGGTCTCCAACAAAGCCC |
| | | TCCCAGCCCCCATCGAGAAAACCATCTCCAAAGCCAAAG |
| | | GGCAGCCCCGAGAACCACAGGTGTACACCCTGCCCCCAT |
| | | CCCGGGATGAGCTGACCAAGAACCAGGTCAGCCTGACCT |
| | | GCCTGGTCAAAGGCTTCTATCCCAGCGACATCGCCGTGG |
| | | AGTGGGAGAGTAATGGGCAGCCGGAGAACAACTACAAG |
| | | ACCACGCCTCCCGTGCTGGACTCCGACGGCTCCTTCTTCC |
| | | TCTACAGCAAGCTCACCGTGGACAAGAGCAGGTGGCAG |
| | | CAGGGGAACGTCTTCTCATGCTCCGTGATGCATGAGGCT |
| | | CTGCACAACCACTACACGCAGAAGAGCCTCTCCCTGTCT |
| | | CCGGGTAAATAA |
| 9 | aa sequence of | GFIFSNHW |
| | Heavy Chain | |
| | CDR1 predicted | |
| | by CDR-IMGT | |
| | of anti-TNFα | |
| | antibody | |
| | (XTMAB-16, | |
| | target antibody) | |
| 10 | aa sequence of | IRSKSINSAT |
| | Heavy Chain | |
| | CDR2 predicted | |
| | by CDR-IMGT | |
| | of anti-TNFα | |
| | antibody | |
| | (XTMAB-16, | |
| | target antibody) | |
| 11 | aa sequence of | SRNYYGSTYDY |
| | Heavy Chain | |
| | CDR3 predicted | |
| | by CDR-IMGT | |
| | of anti-TNFα | |
| | antibody | |
| | (XTMAB-16, | |
| | target antibody) | |
| 12 | aa sequence of | QFVGSS |
| | Light Chain | |
| | CDR1 predicted | |
| | by CDR-IMGT | |
| | of anti-TNFα | |
| | antibody | |
| | (XTMAB-16, | |
| | target antibody) | |
| | aa sequence of | YA |
| | Light Chain | |
| | CDR2 predicted | |
| | by CDR-IMGT | |
| | of anti-TNFα | |
| | antibody | |
| | (XTMAB-16, | |
| | target antibody) | |
| 14 | aa sequence of | QQSHSWPFT |
| | Light Chain | |
| | CDR3 predicted | |
| | by CDR-IMGT | |
| | of anti-TNFα | |
| | antibody | |
| | (XTMAB-16, | |
| | target antibody) | |
| 15 | aa sequence of | NHWMN |
| | Heavy Chain | |
| | CDR1 predicted | |
| | by CDR-Kabat | |
| | of anti-TNFα | |
| | antibody | |
| | (XTMAB-16, | |
| | target antibody) | |
| 16 | aa sequence of | EIRSKSINSATHYAESVKG |
| | Heavy Chain | |
| | CDR2 predicted | |
| | by CDR-Kabat | |
| | of anti-TNFα | |
| | antibody | |
| | (XTMAB-16, | |
| | target antibody) | |
| 17 | aa sequence of | NYYGSTYDY |
| | Heavy Chain | |
| | CDR3 predicted | |
| | by CDR-Kabat | |
| | of anti-TNFα | |
| | antibody | |
| | (XTMAB-16, | |
| | target antibody) | |
| 18 | aa sequence of | RASQFVGSSIH |
| | Light Chain | |
| | CDR1 predicted | |
| | by CDR-Kabat | |
| | of anti-TNFα | |
| | antibody | |
| | (XTMAB-16, | |
| | target antibody) | |
| 19 | aa sequence of | YASESMS |
| | Light Chain | |
| | CDR2 predicted | |
| | by CDR-Kabat | |
| | of anti-TNFα | |
| | antibody | |
| | (XTMAB-16, | |
| | target antibody) | |
| 20 | aa sequence of | QQSHSWPFT |
| | Light Chain | |
| | CDR3 predicted | |
| | by CDR-Kabat | |
| | of anti-TNFα | |
| | antibody | |
| | (XTMAB-16, | |
| | target antibody) | |

### Dissociating NAbs

### Acid Wash

In some embodiments, the method comprises disassociating the NAb from the target by adding an acid wash to the sample. The acid wash may be any acid wash known to one of skill in the art to disassociate an antibody from its target. In some embodiments, the acid wash may comprise acetic acid.

In some embodiments, the acid wash comprises about 2M acetic acid. In some embodiments, the acid wash comprises about 300 mM acetic acid. In some embodiments, the acid wash comprises 0.1M glycine-HCl.

In some embodiments, the acid wash comprises about 10 mM, about 50 mM, about 100 mM, about 150 mM, about 200 mM, about 250 mM, about 300 mM, about 350 mM, about 400 mM, about 500 mM, about 600 mM, about 700 mM, about 800 mM, about 900 mM, about 1M, about 1.5M, about 2M, about 2.5M, about 3M, about 3.5M, about 4M, about 4.5M, or about 5M acetic acid.

In some embodiments, the acid wash comprises at least 10 mM, at least 50 mM, at least 100 mM, at least 150 mM, at least 200 mM, at least 250 mM, at least 300 mM, at least 350 mM, at least 400 mM, at least 500 mM, at least 600 mM, at least 700 mM, at least 800 mM, at least 900 mM, at least 1M, at least 1.5M, at least 2M, at least 2.5M, at least 3M, at least 3.5M, at least 4M, at least 4.5M, or at least 5M acetic acid.

In some embodiments, the acid wash comprises at least about 10 mM, at least about 50 mM, at least about 100 mM, at least about 150 mM, at least about 200 mM, at least about 250 mM, at least about 300 mM, at least about 350 mM, at least about 400 mM, at least about 500 mM, at least about 600 mM, at least about 700 mM, at least about 800 mM, at least about 900 mM, at least about 1M, at least about 1.5M, at least about 2M, at least about 2.5M, at least about 3M, at least about 3.5M, at least about 4M, at least about 4.5M, or at least about 5M acetic acid.

In some embodiments, the acid wash comprises about 0.01M, about 0.02M, about 0.03M, about 0.04M, about 0.05M, about 0.06M, about 0.07M, about 0.08M, about 0.09M, about 0.1M, about 0.2M, about 0.3M, about 0.4M, about 0.5M, about 0.6M, about 0.7M, about 0.8M, about 0.9M, or about 1.0M glycine-HCl.

In some embodiments, the acid wash comprises at least 0.01M, at least 0.02M, at least 0.03M, at least 0.04M, at least 0.05M, at least 0.06M, at least 0.07M, at least 0.08M, at least 0.09M, at least 0.1M, at least 0.2M, at least 0.3M, at least 0.4M, at least 0.5M, at least 0.6M, at least 0.7M, at least 0.8M, at least 0.9M, or at least 1.0M glycine-HCl.

In some embodiments, the acid wash comprises at least about 0.01M, at least about 0.02M, at least about 0.03M, at least about 0.04M, at least about 0.05M, at least about 0.06M, at least about 0.07M, at least about 0.08M, at least about 0.09M, at least about 0.1M, at least about 0.2M, at least about 0.3M, at least about 0.4M, at least about 0.5M, at least about 0.6M, at least about 0.7M, at least about 0.8M, at least about 0.9M, or at least about 1.0M glycine-HCl.

In some embodiments, the acid wash has a pH of about 0.5, about 1.0, about 1.5, about 2.0, about 2.5, about 3.0, about 3.5, or about 4.0.

In some embodiments, the acid wash has a pH of at least 0.5, at least 1.0, at least 1.5, at least 2.0, at least 2.5, at least 3.0, at least 3.5, or at least 4.0.

In some embodiments, the acid wash has a pH of at least about 0.5, at least about 1.0, at least about 1.5, at least about 2.0, at least about 2.5, at least about 3.0, at least about 3.5, or at least about 4.0.

### Purifying NAbs

In some embodiments, the method comprises purifying a NAb. In some embodiments, the NAb is purified using affinity chromatography. In some embodiments, the NAb is purified using a microbead-based purification technology. For example, the NAb may be purified using a magnetic bead purification system. In some embodiments, antibody purification comprises the addition of chaotropic ions.

### Affinity Antibodies

The antibodies of the present technology may be labeled, such as with an affinity label or a detection label. Nonlimiting examples of antibody labels include a biotin label, a fluorescent label, an enzyme label (e.g., horseradish peroxidase (HRP) and alkaline phosphatase (AP)), gold nanoparticles, radioisotopes, and drug molecules.

In some embodiments, the antibodies are conjugated to an additional molecule or compound, forming an antibody conjugate. The conjugation may occur via a covalent linkage. In some embodiments, the antibodies of the present technology are directly (e.g., labeled via conjugation) or indirectly labeled with an affinity label or a detection label. A detection label may be any label that may be detected directly or indirectly. As used herein, “direct” detection of the detection label includes measuring the presence of the detection label or inducing the detection label to produce a detectable substance.

For example, the detection label may be a luminescent compound. In some embodiments, the detection is configured to produce a chemiluminescent or ECL signal in the presence of a marker. In some embodiments, the detection label is an enzyme configured to convert a chromogenic marker into a pigment. For example, the enzyme is a HRP and the chromogenic marker may be 3,3′,5,5′-tetramethylbenzidine (TMB). In some embodiments, the detection label comprises a sulfo-Tag. The sulfo-Tag (Ru-tag) may be contacted with a marker comprising tripropylamine (TPA), and the level of the detection label may be measured by applying an electrical current to the marker in the presence of the detection label to produce the ECL signal.

In some embodiments, the detection label is detected by a meso scale discovery (MSD) assay. In some embodiments, the detection label is detected by ECL. In some embodiments, the detection label is a ruthenium (Ru) metal ion. In some embodiments, the Ru ion is detected when the ion comes in close proximity with an electrode. As used herein, a “close proximity” between the Ru ion and the electrode is a distance between the Ru ion and the electrode that is small enough to cause a detectable oxidation-reduction reaction by ECL as understood by a person of skill in the art. In some embodiments, the solid phase comprises an electrode. In some embodiments, a Ru ion is detected by a charge-coupled device (CCD) camera when it is in close proximity with the electrode.

In some embodiments, the detection label is detected indirectly. As used herein, “indirect” detection of the detection antibody includes adding a separate component that interacts with the detection label, where the separate component is capable of being detected. For example, a secondary antibody may be added, where the secondary antibody is conjugated to a detectable luminescent probe or enzyme configured to convert a chromogenic substrate into a pigment (e.g., a detection antibody conjugated to a detection label).

An affinity label may be any molecular label that has affinity with the second affinity label. In some embodiments, a first affinity label is configured to make a covalent bond with the second affinity label. For example, the first affinity label may be biotin and the second affinity label may be streptavidin or vice versa. The first and second affinity labels may be any pairing of affinity labels known to one of ordinary skill in the art. In some embodiments, the first affinity label may be glutathione and the second affinity label may be glutathione S-transferase or vice versa. In some embodiments, the first affinity label may be maltose and the second affinity label may be maltose-binding protein or vice versa. In some embodiments, the first affinity label is chitin and the second affinity label is chitin-binding protein or vice versa.

In some embodiments, the detection label may be a third affinity label and detecting the detection label further comprises contacting the third affinity label with a fourth affinity label conjugated to a detectable compound, where the third affinity label binds to the fourth affinity label. For example, the fourth affinity label may be conjugated to a luminescent compound or enzyme configured to convert a chromogenic substrate into a pigment.

The third affinity label may be any molecular label that has affinity with the fourth affinity label. In some embodiments, the third affinity label is configured to make a covalent bond with the fourth affinity label. For example, the third affinity label may be biotin and the fourth affinity label may be streptavidin or vice versa. The third and fourth affinity labels may be any pairing of affinity labels known to one of ordinary skill in the art. In some embodiments, the third affinity label may be glutathione and the fourth affinity label may be glutathione S-transferase or vice versa. In some embodiments, the third affinity label may be maltose and the fourth affinity label may be maltose-binding protein or vice versa. In some embodiments, the third affinity label is chitin and the fourth affinity label is chitin-binding protein or vice versa.

### Methods of Purification

In some embodiments, purifying the NAb comprises contacting the sample with an anti-TNFα antibody conjugated to a first affinity label (i.e., “affinity antibody”) and contacting the sample containing the NAb and the affinity antibody with a first solid phase coated with a second affinity label. In some embodiments, the affinity antibody is added at a concentration of about 10,000 ng/mL-20,000 ng/mL, about 20,000 ng/mL-30,000 ng/mL, about 30,000 ng/mL-40,000 ng/mL, or about 40,000 ng/mL-50,000 ng/mL. In some embodiments, the affinity antibody is added at a concentration of about 30,000 ng/mL.

In some embodiments, the affinity antibody is added at a concentration of at least 10,000 ng/mL-20,000 ng/mL, at least 20,000 ng/mL-30,000 ng/mL, at least 30,000 ng/mL-40,000 ng/mL, or at least 40,000 ng/mL-50,000 ng/mL. In some embodiments, the affinity antibody is added at a concentration of at least 30,000 ng/mL.

In some embodiments, the affinity antibody is added at a concentration of at least about 10,000 ng/mL-20,000 ng/mL, at least about 20,000 ng/mL-30,000 ng/mL, at least about 30,000 ng/mL-40,000 ng/mL, or at least about 40,000 ng/mL-50,000 ng/mL. In some embodiments, the affinity antibody is added at a concentration of at least about 30,000 ng/mL.

As used herein, “solid phase” may be any medium that remains stationary or temporarily stationary as a mobile phase moves across or through the solid phase, such that components of the mobile phase may interact with the solid phase. A solid phase may include a plate, chromatography column, microbead, magnetic microbead, or any other solid phase known to one of ordinary skill in the art. In some embodiments, the first solid phase is a microbead, plate, or column. In some embodiments, the first solid phase is a microbead. In some embodiments, the first solid phase is a magnetic microbead.

In some embodiments, purifying the NAb comprises contacting the sample with the affinity antibody; contacting the sample containing the NAb and the affinity antibody with a first solid phase coated with a second affinity label; and washing the first solid phase. In some embodiments, the solid phase is washed with a wash buffer that does not disrupt the interaction between the NAb and the affinity antibody. In some embodiments, the wash buffer comprises phosphate-buffered saline (PBS). In some embodiments, the wash buffer comprises a phosphate-buffered solution with a surfactant. In some embodiments, the wash buffer comprises Tween-20. In some embodiments, the wash buffer comprises 1×PBS with about 0.05% Tween-20. In some embodiments, the wash buffer comprises about 50 mM Tris. In some embodiments, the wash buffer comprises about 0.14 M NaCl. In some embodiments, the wash buffer has a pH of about 8.0. In some embodiments, the wash buffer comprises about 50 mM Tris, about 0.14 M NaCl, about 0.05% Tween-20, and has a pH of about 8.0.

In some embodiments, purifying the NAb comprises contacting the sample with the affinity antibody; contacting the sample containing the NAb and the affinity antibody with a first solid phase coated with a second affinity label; washing the first solid phase; and eluting the NAb from the first solid phase. In some embodiments, the NAb is eluted using an elution buffer that disrupts the interaction between the NAb and the affinity antibody. In some embodiments, the elution buffer comprises glycine and sodium chloride (NaCl). In some embodiments, the elution buffer comprises about 0.1M glycine and about 0.15M NaCl. In some embodiments, the elution buffer is acidic. In some embodiments, the elution buffer has a pH of about 2 to about 3. In some embodiments, the elution buffer has a pH of about 2.2. In some embodiments, the elution buffer has a pH of about 2.8.

In some embodiments, the NAb purification is performed using an automated purification system. As used herein, an “automated purification system” refers to a system that purifies an antibody according to steps that are in part performed by a robotic or mechanical platform. The automated purification system may be completely automated or partially automated. In some embodiments, a human “loads” the automated purification system with the samples and reagents of the present technology and the automated purification system performs the remaining steps. For example, the automated purification system may be a KingFisher™ Flex Purification System or another automated purification system that uses magnetic particle separation technology. Other automated purification systems having at least the foregoing features and known to one of skill in the art are within the scope of this technology.

The method further comprises contacting the purified NAb with the detection antibody, which is an anti-TNFα antibody conjugated to a detection label. The detection label may be any label that may be detected directly or indirectly. As used herein, “direct” detection of the detection label includes measuring the presence of the detection label or inducing the detection label to produce a detectable substance. For example, the detection label may be a luminescent compound. In some embodiments, the detection label comprises an enzyme configured to convert a chromogenic substrate into a pigment. For example, the enzyme is a horseradish peroxidase (HRP) and the chromogenic substrate may be 3,3′,5,5′-tetramethylbenzidine (TMB). In some embodiments, the detection label is detected by a meso scale discovery (MSD) assay. In some embodiments, the detection label is detected by electrochemiluminescence. In some embodiments, the detection label is a ruthenium (Ru) metal ion. In some embodiments, the Ru ion is detected when the ion comes in close proximity with an electrode. As used herein, a “close proximity” between the Ru ion and the electrode is a distance between the Ru ion and the electrode that is small enough to cause a detectable oxidation-reduction reaction by electrochemiluminescence as understood by a person of skill in the art. In some embodiments, the TNFα-coated substrate comprises an electrode. In some embodiments, a Ru ion is detected by a CCD camera when it is in close proximity with the electrode.

In some embodiments, the detection antibody is added at a concentration of about 10,000 ng/mL-20,000 ng/mL, about 20,000 ng/mL-30,000 ng/mL, about 30,000 ng/mL-40,000 ng/mL, or about 40,000 ng/mL-50,000 ng/mL. In some embodiments, the detection antibody is added at a concentration of about 30,000 ng/mL.

In some embodiments, the detection antibody is added at a concentration of at least 10,000 ng/mL-20,000 ng/mL, at least 20,000 ng/mL-30,000 ng/mL, at least 30,000 ng/mL-40,000 ng/mL, or at least 40,000 ng/mL-50,000 ng/mL. In some embodiments, the detection antibody is added at a concentration of at least 30,000 ng/mL.

In some embodiments, the detection antibody is added at a concentration of at least about 10,000 ng/mL-20,000 ng/mL, at least about 20,000 ng/mL-30,000 ng/mL, at least about 30,000 ng/mL-40,000 ng/mL, or at least about 40,000 ng/mL-50,000 ng/mL. In some

The method of the present technology comprises contacting the purified NAb and the anti-TNFα antibody conjugate with a TNFα-coated substrate. The TNFα-coated substrate may comprise any substrate coated with TNFα by methods known to one of ordinary skill in the art. In some embodiments, the substrate is coated with human TNFα.

The substrate may be coated with about 50 μg/mL-200 μg/mL, about 75 μg/mL-150 μg/mL, or about 90 μg/mL-110 μg/mL TNFα. In some embodiments, the substrate is coated with about 100 μg/mL TNFα.

The substrate may be coated with at least 50 μg/mL-200 μg/mL, at least 75 μg/mL-150 μg/mL, or at least 90 μg/mL-110 μg/mL TNFα. In some embodiments, the substrate is coated with at least 100 μg/mL.

The substrate may be coated with at least about 50 μg/mL-200 μg/mL, at least about 75 μg/mL-150 μg/mL, or at least about 90 μg/mL-110 μg/mL TNFα. In some embodiments, the substrate is coated with at least about 100 μg/mL.

In some embodiments, the TNFα substrate is blocked to prevent non-specific binding to the substrate. The TNFα substrate may be blocked using any blocking buffer or reagent known to one of ordinary skill in the art. In some embodiments, the substrate is a plate.

In some embodiments, after the purified NAb and detection antibody are contacted with the TNFα-coated substrate, the substrate is washed. In some embodiments, the TNFα-coated substrate is washed with a wash buffer that does not disrupt interactions between the NAb and the TNFα-coated substrate or interactions between the NAb and the detection antibody. The wash buffer may be configured to remove the NAb that binds the detection antibody from the substrate, but it does not remove the NAb that binds the TNFα-coated substrate. In some embodiments, the substrate is a plate.

In some embodiments, the method further comprises detecting the detection label. In some embodiments, the detection label is detected by measuring a luminescent compound. In some embodiments, the detection label is detected by inducing an enzyme to convert a chromogenic substrate into the pigment and then detecting the pigment and measuring the presence of the pigment. In some embodiments, detecting the detection label comprises converting the chromogenic substrate into the pigment and detecting the optical density (OD) of the sample. In some embodiments, the detection label is a Ru metal ion and detecting the detection label comprises using a camera to detect light emitted by a Ru metal ion that is in close proximity to a TNFα-coated substrate comprising an electrode.

The method further comprises detecting the NAb when the detection antibody binds the NAb instead of the TNFα-coated substrate. When the detection antibody binds an NAb instead of the TNFα-coated substrate, then less of the detection antibody will bind to the substrate, and therefore the detection label will be lower when the NAb is present. In some embodiments, the substrate is a plate. In embodiments where the detection label is detected with a luminescent compound, the presence of the NAb may be measured by lower luminescent intensity. In embodiments where the detection label is detected with a pigment, the presence of the NAb may be measured by a lower density of pigment. In embodiments where the detection label is detected with a pigment, the presence of the NAb may be measured by a higher OD (i.e., more light passes through the sample because there is less pigment). In embodiments where the detection label is an Ru-metal ion, the presence of the NAb may be measured by a higher intensity of light emitted when the Ru-metal ion is in close proximity to the TNFα-coated substrate.

In some embodiments, the NAb is detected by determining whether the detection label is present at a level below a predetermined level. In some embodiments, the NAb is detected by determining that the OD of the sample is above a predetermined cut-point.

### Control Samples

In some embodiments, the method further comprises performing the method on a negative control sample. In some embodiments, the negative control sample has no detectable NAb. In some embodiments, the negative control sample is a drug-naïve sample from a healthy subject who has not received the target antibody. In some embodiments, the method is performed on the negative control sample in parallel with the sample. As used herein, performing the method “in parallel” includes performing the method on the negative control sample at the same time and/or on the same plate as the sample. Performing the method on the negative control sample comprises (i) treating the negative control sample with an acid wash; (ii) performing the purification steps on the negative control sample; (iii) contacting the negative control sample with the detection antibody; (iv) contacting the negative control sample having the detection antibody with a TNFα-coated substrate; and (v) detecting the absence of NAb by measuring anti-TNFα antibody conjugate in contact with the TNFα-coated substrate.

In some embodiments, the method further comprises performing the method on a positive control sample. In some embodiments, the positive control sample is a sample from a subject who has received the target antibody. In some embodiments, the positive control sample is a sample that has been shown to contain NAbs in a previous assay. The method may be performed on the positive control sample in parallel with the sample. The method may be performed on a sample, a negative control sample, and a positive control sample in parallel.

In some embodiments, the cut-point is determined by performing the method on a plurality of negative control samples. In some embodiments, the negative control samples are drug-naïve samples from healthy subjects who have never received the target antibody. In some embodiments, the cut-point is determined by performing the method on at least 50 negative control samples. In some embodiments, the OD signal from the 50 negative control samples is normalized by the mean of all negative control samples. In some embodiments, outliers are identified and removed. In some embodiments, the cut-point is identified based on the 0.5%-1%, 1%-1.5%, 1.5%-2%, 2%-2.5%, or 2.5%-3% false-positive rate. In some embodiments, the cut-point is identified based on the 3% false-positive rate. In some embodiments, the cut-point is identified based on the 1% false-positive rate.

In some embodiments, the method further comprises performing the method on a quality control (QC) sample. One or more QC samples may be included to assess the performance of the method. In some embodiments, the QC sample is a blank sample, matrix spike sample (i.e., a plasma or serum sample spiked with NAb), field duplicate sample, replicate sample, and/or reference material sample. The method may be performed on the QC sample in parallel with the sample. The method may be performed on a sample, a negative control sample, a positive control sample, and a QC sample in parallel.

In some embodiments, the predetermined cut-point is an OD of 0.5-2, 0.5-1, 1-1.5, or 1.5-2. In some embodiments, the predetermined cut-point is about 1. In some embodiments, the predetermined cut-point is about 0.91.

### Sensitivity and Target Tolerance

In some embodiments, the sensitivity of detecting the NAb is less than about 150 ng/mL, less than about 200 ng/mL, less than about 250 ng/mL, less than about 300 ng/mL, less than about 350 ng/mL, less than about 400 ng/mL, less than about 450 ng/mL, less than about 500 ng/mL, less than about 550 ng/mL, or less than about 600 ng/mL. In some embodiments, the sensitivity of detecting the NAb is less than about 500 ng/mL. In some embodiments, the sensitivity of detecting the NAb is less than about 250 ng/mL.

In some embodiments, the sensitivity of detecting the NAb is less than at least 150 ng/mL, less than at least 200 ng/mL, less than at least 250 ng/mL, less than at least 300 ng/mL, less than at least 350 ng/mL, less than at least 400 ng/mL, less than at least 450 ng/mL, less than at least 500 ng/mL, less than at least 550 ng/mL, or less than at least 600 ng/mL. In some embodiments, the sensitivity of detecting the NAb is less than at least 500 ng/mL. In some embodiments, the sensitivity of detecting the NAb is less than at least 250 ng/mL.

In some embodiments, the sensitivity of detecting the NAb is less than at least about 150 ng/mL, less than at least about 200 ng/mL, less than at least about 250 ng/mL, less than at least about 300 ng/mL, less than at least about 350 ng/mL, less than at least about 400 ng/mL, less than at least about 450 ng/mL, less than at least about 500 ng/mL, less than at least about 550 ng/mL, or less than at least about 600 ng/mL. In some embodiments, the sensitivity of detecting the NAb is less than at least about 500 ng/mL. In some embodiments, the sensitivity of detecting the NAb is less than at least about 250 ng/mL.

In some embodiments, the target tolerance of detecting the NAb is about 500 ng/mL-750 ng/mL, about 750 ng/mL-1000 ng/mL, about 1000 ng/mL-1250 ng/mL, or about 1250 ng/mL-1500 ng/mL. In some embodiments, the target tolerance of detecting the NAb is about 1000 ng/mL.

In some embodiments, the target tolerance of detecting the NAb is at least 500 ng/mL-750 ng/mL, at least 750 ng/mL-1000 ng/mL, at least 1000 ng/mL-1250 ng/mL, or at least 1250 ng/mL-1500 ng/mL. In some embodiments, the target tolerance of detecting the NAb is at least 1000 ng/mL.

In some embodiments, the target tolerance of detecting the NAb is at least about 500 ng/mL-750 ng/mL, at least about 750 ng/mL-1000 ng/mL, at least about 1000 ng/mL-1250 ng/mL, or at least about 1250 ng/mL-1500 ng/mL. In some embodiments, the target tolerance of detecting the NAb is at least about 1000 ng/mL.

### Kits

The present technology further comprises a kit for detecting an NAb against the target antibody comprising the affinity antibody; a first solid phase coated with a second affinity label; the detection antibody; a substrate; and TNFα. The concentration of the reagents in the kit is formulated to enable a person of ordinary skill in the art to perform the methods of the present technology.

In some embodiments, the affinity antibody comprises a first affinity label. In some embodiments, the first affinity label is biotin. In some embodiments, the detection antibody comprises a detection label. In some embodiments, the detection label is a luminescent compound, an enzyme configured to convert a chromogenic substrate into a pigment, or biotin. In some embodiments, the detection label is biotin.

In some embodiments, the kit comprises a negative control. The negative control may be a sample from a drug-naïve subject. In some embodiments, the negative control is a sample that is known to contain no NAb. In some embodiments, the kit comprises a positive control. The positive control may be a sample from a subject that has received the target antibody. In some embodiments, the positive control is a sample known to contain NAb. In some embodiments, the kit may comprise NAb or NAb solution and instructions for making a positive control. For example, the kit may comprise a NAb solution and instructions to “spike” a plasma or serum sample with the NAb solution to prepare the positive control.

In some embodiments, the kit comprises a “high” positive control (HPC) and a “low” positive control (LPC), where the HPC has a higher concentration than the LPC. For example, the HPC may have a concentration of about 300 ng/mL-400 ng/mL, about 400 ng/mL-500 ng/mL, about 500 ng/mL-600 ng/mL, about 600 ng/mL-700 ng/mL, about 700 ng/mL-800 ng/mL, about 800 ng/mL-900 ng/mL, about 900 ng/mL-1000 ng/mL, or greater than about 1000 ng/mL. The concentration of the LPC may be less than about 1 ng/mL, about 1 ng/mL-10 ng/mL, about 10 ng/mL-100 ng/mL, about 100 ng/mL-200 ng/mL, about 200 ng/mL-300 ng/mL, or about 300 ng/mL-500 ng/mL.

In some embodiments, the HPC has a concentration of at least 300 ng/mL-400 ng/mL, at least 400 ng/mL-500 ng/mL, at least 500 ng/mL-600 ng/mL, at least 600 ng/mL-700 ng/mL, at least 700 ng/mL-800 ng/mL, at least 800 ng/mL-900 ng/mL, at least 900 ng/mL-1000 ng/mL, or greater than at least 1000 ng/mL. The concentration of the LPC may be less than at least 1 ng/mL, at least 1 ng/mL-10 ng/mL, at least 10 ng/mL-100 ng/mL, at least 100 ng/mL-200 ng/mL, at least 200 ng/mL-300 ng/mL, or at least 300 ng/mL-500 ng/mL.

In some embodiments, the HPC has a concentration of at least about 300 ng/mL-400 ng/mL, at least about 400 ng/mL-500 ng/mL, at least about 500 ng/mL-600 ng/mL, at least about 600 ng/mL-700 ng/mL, at least about 700 ng/mL-800 ng/mL, at least about 800 ng/mL-900 ng/mL, at least about 900 ng/mL-1000 ng/mL, or greater than at least about 1000 ng/mL. The concentration of the LPC may be less than at least about 1 ng/mL, at least about 1 ng/mL-10 ng/mL, at least about 10 ng/mL-100 ng/mL, at least about 100 ng/mL-200 ng/mL, at least about 200 ng/mL-300 ng/mL, or at least about 300 ng/mL-500 ng/mL.

The affinity antibody and/or the detection antibody may be affinity purified. In some embodiments, the affinity antibody and/or the detection antibody may be stored as a lyophilized powder. In some embodiments, the affinity antibody and/or detection antibody may be stored as a reconstituted liquid antibody.

Any of the components of the kit may be provided at a concentration that is more concentrated than the concentration at which it is used. For example, the components of the kit may be provided as a “stock solution” that must be diluted to the desired concentration of the “working solution.” In some embodiments, the stock solution may be about 2×, 5×, 10×, 50×, 100×, 500×, 1000×, 5000×, or 10000× more concentrated than the working solution.

The affinity antibody and/or the detection antibody may be lyophilized using methods known to one of skill in the art. One or more lyophilizers may be included. Examples of suitable lyophilizers include, for example, Tris-EDTA and sucrose. Any other lyophilizer commonly used in the art is also available. Determining the appropriate lyophilizer as well as the effective amount of other lyophilizers is within the scope of routine skill in the art.

The kit may include about 50 μg-100 μg, about 100 μg-500 μg, about 500 μg-1000 μg, about 1 μg-10 mg, about 10 μg-50 mg, about 50 μg-100 mg, about 100 μg-500 mg, or about 500 μg-1000 mg of the affinity antibody and/or the detection antibody. The kit may include about 50 μg-200 μg of the affinity antibody and/or the detection antibody. The kit may include about 100 mg of the affinity antibody and/or the detection antibody. The kit may include about 1 mg of the affinity antibody and/or the detection antibody. The purity of the affinity antibody and/or the detection antibody may be about 90%, 91%, 92%, 93%, 94%, 95%, 96%, 97%, 98%, 99%, or greater than 99%.

The kit may include at least 50 μg-100 μg, at least 100 μg-500 μg, at least 500 μg-1000 μg, at least 1 μg-10 mg, at least 10 μg-50 mg, at least 50 μg-100 mg, at least 100 μg-500 mg, or at least 500 μg-1000 mg of the affinity antibody and/or the detection antibody. The kit may include at least 50 μg-200 μg of the affinity antibody and/or the detection antibody. The kit may include at least 100 mg of the affinity antibody and/or the detection antibody. The kit may include at least 1 mg of the affinity antibody and/or the detection antibody. The purity of the affinity antibody and/or the detection antibody may be at least 90%, 91%, 92%, 93%, 94%, 95%, 96%, 97%, 98%, 99%, or greater than 99%.

The kit may include at least about 50 μg-100 μg, at least about 100 μg-500 μg, at least about 500 μg-1000 μg, at least about 1 μg-10 mg, at least about 10 μg-50 mg, at least about 50 μg-100 mg, at least about 100 μg-500 mg, or at least about 500 μg-1000 mg of the affinity antibody and/or the detection antibody. The kit may include at least about 50 μg-200 μg of the affinity antibody and/or the detection antibody. The kit may include at least about 100 mg of the affinity antibody and/or the detection antibody. The kit may include at least about 1 mg of the affinity antibody and/or the detection antibody. The purity of the affinity antibody and/or the detection antibody may be at least about 90%, 91%, 92%, 93%, 94%, 95%, 96%, 97%, 98%, 99%, or greater than 99%.

In some embodiments, the lyophilized affinity antibody and/or detection antibody may be reconstituted in about 50 μg-100 μL, about 100 μg-150 μL, about 150 μg-200 μL, about 200 μg-250 μL, about 250 μg-300 μL, about 300 μg-350 μL, about 350 μg-400 μL, about 400 μg-450, about 350 μg-400 μL, about 400 μg-450 μL, or about 450 μg-500 μL of a reconstitution buffer.

In some embodiments, the lyophilized affinity antibody and/or detection antibody may be reconstituted in at least 50 μg-100 μL, at least 100 μg-150 μL, at least 150 μg-200 μL, at least 200 μg-250 μL, at least 250 μg-300 μL, at least 300 μg-350 μL, at least 350 μg-400 μL, at least 400 μg-450, at least 350 μg-400 μL, at least 400 μg-450 μL, or at least 450 μg-500 μL of a reconstitution buffer.

In some embodiments, the lyophilized affinity antibody and/or detection antibody may be reconstituted in at least about 50 μg-100 μL, at least about 100 μg-150 μL, at least about 150 μg-200 μL, at least about 200 μg-250 μL, at least about 250 μg-300 μL, at least about 300 μg-350 μL, at least about 350 μg-400 μL, at least about 400 μg-450, at least about 350 μg-400 μL, at least about 400 μg-450 μL, or at least about 450 μg-500 μL of a reconstitution buffer.

The reconstitution buffer may comprise any components known to one of skill in the art. For example, the reconstitution buffer may comprise water, PBS, DMSO, glycerol, or sodium azide.

The reconstitution buffer may comprise about 10%-15%, about 15%-20%, about 20%-25%, about 25%-30%, or about 30%-35% glycerol. The reconstitution buffer may comprise about 0.02%-0.04%, about 0.04%-0.06%, about 0.06%-0.08%, about 0.08%-0.1%, about 0.1%-0.12%, about 0.12%-0.14%, or about 0.14%-0.16% sodium azide. In some embodiments, the reconstitution buffer has a pH of about 6 to about 7 or about 7 to about 8. In some embodiments, the reconstitution buffer has a pH of about 7.2.

The reconstitution buffer may comprise at least 10%-15%, at least 15%-20%, at least 20%-25%, at least 25%-30%, or at least 30%-35% glycerol. The reconstitution buffer may comprise at least 0.02%-0.04%, at least 0.04%-0.06%, at least 0.06%-0.08%, at least 0.08%-0.1%, at least 0.1%-0.12%, at least 0.12%-0.14%, or at least 0.14%-0.16% sodium azide. In some embodiments, the reconstitution buffer has a pH of at least 6 to at least 7 or at least 7 to at least 8. In some embodiments, the reconstitution buffer has a pH of at least 7.2.

The reconstitution buffer may comprise at least about 10%-15%, at least about 15%-20%, at least about 20%-25%, at least about 25%-30%, or at least about 30%-35% glycerol. The reconstitution buffer may comprise at least about 0.02%-0.04%, at least about 0.04%-0.06%, at least about 0.06%-0.08%, at least about 0.08%-0.1%, at least about 0.1%-0.12%, at least about 0.12%-0.14%, or at least about 0.14%-0.16% sodium azide. In some embodiments, the reconstitution buffer has a pH of at least about 6 to at least about 7 or at least about 7 to at least about 8. In some embodiments, the reconstitution buffer has a pH of at least about 7.2.

In some embodiments, the kit comprises a reconstitution buffer. In some embodiments, the kit comprises an affinity antibody and/or detection antibody that is reconstituted.

The kit may include about 1 μg/mL-10 μg/mL, about 10 μg/mL-50 μg/mL, about 50 μg/mL-100 μg/mL, about 100 μg/mL-500 μg/mL, about 500 μg/mL-1000 μg/mL, about 1 mg/mL-10 mg/mL, about 10 mg/mL-50 mg/mL, about 50 mg/mL-100 mg/mL, about 100 mg/mL-1000 mg/mL, or about 1000 mg/mL-10000 mg/mL of the detection antibody and/or affinity antibody in solution.

The kit may include at least 1 μg/mL-10 μg/mL, at least 10 μg/mL-50 μg/mL, at least 50 μg/mL-100 μg/mL, at least 100 μg/mL-500 μg/mL, at least 500 μg/mL-1000 μg/mL, at least 1 mg/mL-10 mg/mL, at least 10 mg/mL-50 mg/mL, at least 50 mg/mL-100 mg/mL, at least 100 mg/mL-1000 mg/mL, or at least 1000 mg/mL-10000 mg/mL of the detection antibody and/or affinity antibody in solution.

The kit may include at least about 1 μg/mL-10 μg/mL, at least about 10 μg/mL-50 μg/mL, at least about 50 μg/mL-100 μg/mL, at least about 100 μg/mL-500 μg/mL, at least about 500 μg/mL-1000 μg/mL, at least about 1 mg/mL-10 mg/mL, at least about 10 mg/mL-50 mg/mL, at least about 50 mg/mL-100 mg/mL, at least about 100 mg/mL-1000 mg/mL, or at least about 1000 mg/mL-10000 mg/mL of the detection antibody and/or affinity antibody in solution.

The first solid phase may include any solid phase known to one of skill in the art to be useful for antibody affinity purification. In some embodiments, the first solid phase is a microbead. In some embodiments, the first solid phase is a magnetic microbead. The kit may include 1 μg/mL-10 μg/mL, 10 μg/mL-50 μg/mL, 50 μg/mL-100 μg/mL, 100 μg/mL-500 μg/mL, or 500 μg/mL-1000 μg/mL, 1 mg/mL-10 mg/mL, 10 mg/mL-50 mg/mL, 50 mg/mL-100 mg/mL, 100 mg/mL-1000 mg/mL, or 1000 mg/mL-10000 mg/mL of microbeads in solution. The kit may include 0.5 mL-1 mL, 1 mL-1.5 mL, 1.5 mL-2 mL, 2 mL-2.5 mL, 2.5 mL-3 mL, 3 mL-3.5 mL, 3.5 mL-4 mL, 4 mL-4.5 mL, or 4.5 mL-5 mL of microbeads in solution.

In some embodiments, the second affinity label is streptavidin. In some embodiments, the kit comprises a solid phase, a second affinity label, and instructions for coating the solid phase with the second affinity label.

The substrate may be any material known to one of skill that is imageable and capable of binding TNFα. For example, the substrate may be a slide, plate, dish, or flask. In some embodiments, the kit comprises a substrate that is coated with TNFα. In some embodiments, the kit comprises a substrate, TNFα, and instructions for coating the substrate with TNFα. For example, the kit may comprise about 1 μg-5 μg, 5 μg-10 μg, 10 μg-20 μg, or 20 μg-50 μg of human TNFα. In some embodiments, the TNFα is a 1 ng/mL-10 ng/mL, 10 ng/mL-100 ng/mL, 100 ng/mL-1000 ng/mL, or 1000 ng/mL-10000 ng/mL solution of human TNFα. The TNFα solution may comprise any media known to one of skill in the art, for example phosphate buffered saline (PBS).

The kit may comprise one or more buffers. In some embodiments, the kit comprises an acid wash buffer. In some embodiments, the kit further comprises a washing buffer. In some embodiments, the kit further comprises an elution buffer. The buffers of the present technology may be formulated according to any of the formulations of this technology or the formulations known to one of skill in the art.

In accordance with some embodiments, the kit may contain one or more consumables for performing the methods of the present technology. For example, the kit may include one or more plates containing a plurality of wells. In some embodiments, the plate may include 6, 9, 12, 16, 24, 36, 96, 384, or 1536 wells. In some embodiments, a kit or system comprises 1, 2, 3, 4, 5, 6, 7, 8, 9, 10, 11, 12, 13, 14, 15, 16, 17, 18, 19, 20, or more than 20 assay plates.

The kit may also include instructions for performing all or part of the methods described in the present technology. In some embodiments, the kit comprises links (e.g., URLs or QR codes) that direct a user to instructions for performing all or part of the methods of the present technology. The kit may include software or other computer technology to facilitate performing the methods of the present technology.

The components of the kit may be stored to provide stability to the particular components. For example, components of the kit may be stored in a light-impermeable container if the component comprises a light-sensitive compound. In some embodiments, the components of the kit may be stored in an airtight container. In some embodiments, the components of the kit are stored frozen, in liquid form, or in solid form. In some embodiments, the components of the kit are stored at about −195° C., −80° C., −20° C., 4° C., or 20° C.

In another embodiment, the kit comprises one or more of the components, consumables, or reagents described in the examples below.

While the present technology is capable of being embodied in various forms, the description below of several embodiments is made with the understanding that the present disclosure is to be considered as an exemplification of the present technology and is not intended to limit the present technology to the specific embodiments illustrated. Headings are provided for convenience only and are not to be construed to limit the present technology in any manner. Embodiments illustrated under any heading may be combined with embodiments illustrated under any other heading.

From the foregoing, it will be appreciated that specific embodiments of the present technology have been described herein for purposes of illustration, but that various modifications may be made without deviating from the scope of the present technology. Accordingly, the present technology is not limited except as by the appended claims.

### EXAMPLES

### Abbreviations

ADA: Anti-Drug Antibody; A.T.: Ambient Temperature; Bio-MAB-16: Biotin Labeled MAB-16; CP: Cut-Point; Conc.: Concentration; % CV: Percent Coefficient of Variation; diH₂O: Deionized Water; ECL: Electrochemiluminescence; ECLIA: Electrochemiluminescence Immunoassay; FT: Freeze/Thaw: HPC: High Positive Control; LPC: Low Positive Control; MSD: Meso Scale Discovery; NA: Not Applicable; NC: Negative Control; pNHS: Pooled Naïve Human Serum; PBS: Phosphate-Buffered Saline; QC: Quality Control; rECL: Relative ECL; Ru-MAB-16: Ruthenium Labeled MAB-16; SCP: Screening Cut-Point; S-CPF: Screening Cut-Point Factor; SD: Standard Deviation; TCP: Titration Cut-Point; T-CPF: Titration Cut-Point Factor.

### Overview

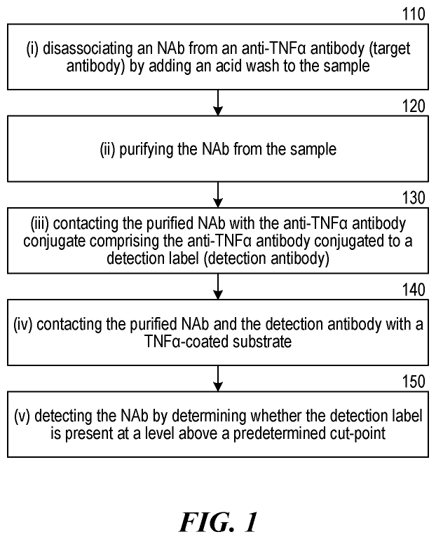

FIG. 1 illustrates a method for detecting an NAb against the target antibody in the sample comprises (i) disassociating the NAb from the target antibody by adding an acid wash to the sample 110; (ii) purifying the NAb from the sample 120; (iii) contacting the purified NAb with an anti-TNFα antibody conjugate comprising the anti-TNFα antibody conjugated to a detection label (detection antibody) 130; (iv) contacting the purified NAb and the detection antibody with a TNFα-coated substrate 140; and (v) detecting the NAb by determining whether the detection label is present at a level above a predetermined cut-point 150.

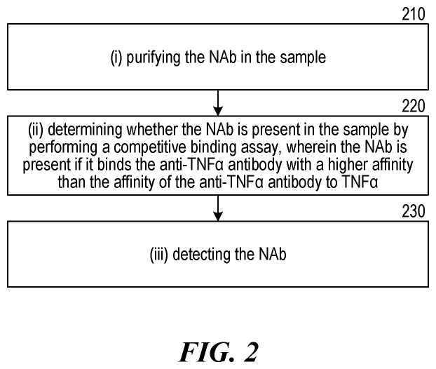

FIG. 2 illustrates a method for detecting an NAb against the target antibody comprises (i) purifying the NAb in the sample 210; (ii) determining whether the NAb is present in the sample by performing a competitive binding assay, wherein the NAb is present if it binds the detection antibody with a higher affinity than a binding affinity of the detection antibody to TNFα 220; and (iii) detecting the NAb 230; wherein a sensitivity of the method is 250 ng/mL or less.

### Example 1: Methods for Detecting NAbs

The presence of NAbs against XTMAB-16 in human serum based on competitive ligand binding (CLB) assay was assessed. XTAMB-16 is an anti-TNFα monoclonal antibody for the treatment of patients with sarcoidosis. XTMAB-16 is an example of an anti-TNFα chimeric human-murine monoclonal IgG1-kappa antibody comprising SEQ ID NO: 1 and 4 (i.e., a “target antibody”) of the present technology.

### Buffers and Solutions

Assay buffer: 33.3 mL or 30% BSA combined with 5 mL of 10% Tween-20 with approximately 800 mL of DPBS. Q.S. (quantum satis) solution to 1.0 L with DPBS. Stored at 2° C.-8° C. for up to 6 months from the date of preparation.

Dilution/wash buffer: C16.7 mL of 30% BSA, 100 mL of 10×DPBS (pH7.4±0.1), 10 mL of 10% Tween-20, 0.5 mL of ProClin 300 combined with approximately 800 mL of deionized water until dissolved. Q.S. solution to 1.0 L with deionized water. Stored at 2° C.-8° C. for up to 6 months from the date of preparation.

Biotin-XTMAB-16 working solution (30,000 ng/mL) in 2M Tris-HCl: Diluted Biotin-XTMAB-16 stock solution to 30,000 ng/mL in 2M Tris-HCl, pH 9.6.

Coating solution (100 ng/mL of Human TNFα in DPBS): Diluted Human TNFα to 100 ng/mL in DPBS.

Neutralizing buffer: Added 1 mL of 1M HEPES (pH 9.0) to 23 mL of Dilution/wash buffer.

Stop solution (1M Phosphoric acid solution): Diluted 68.0 mL 85% phosphoric acid to 1.0 L with diH₂O.

### Preparing Plate Controls

Preparation of Negative Control (NC): NC is pNHS.

Preparation of High Positive Controls (HPC): Anti-XTMAB-16 antibody (91F9D4, 0.781 mg/mL) was used as NAb PC. The concentration of HPC was 600 ng/mL. The final concentration of LPC (220.6 ng/mL) was prepared at 99% confidence interval (1% failure rate) after the SCP was calculated.

TABLE 2
NAb concentrations for controls

| | NAb | Stock | | Total | | Resulting |
|---|---|---|---|---|---|---|
| | Conc. | Vol. | NC Vol. | Vol. | Resulting | Conc. |
| | (ng/ml) | (μL) | (μL) | (μL) | Tube | (ng/mL) |
| Stock | 781,000.0 | 5.0 | 73.1 | 78.1 | PC Int-A | 50000.0 |
| PC Int-A | 50,000.0 | 6.0 | 494.0 | 500.0 | HPC | 600.0 |
| HPC | 600.0 | 36.8 | 63.2 | 100 | LPC | 220.6 |

### Analytical Procedure

On day 1, well plates were coated with TNFα (100 μg/mL) and incubated at 2° C.-8° C. for 16 to 48 hours. On day 2, controls and samples were added to their respective wells. Dilution/wash buffer and acid wash buffer (2M Acetic Acid) were added to wells. Plates were shaken at A.T. for 30 minutes.

Add Biotin-XTMAB-16 working solution was added to each well (30,000 ng/mL) and allowed to shake at A.T. for 60 minutes.

Streptavidin Magnetic Beads Solution was added to wells and plate shook at A.T. for 60 minutes. Dilution/wash buffer was added to wells of four additional well plates (further referred to as Wash 1, 2, 3, and 4 Plates).

0.1M Glycine, 0.15M NaCl, pH 2.2 (Elution Solution) was added into all wells of the well plates (further referred to as Acid Elution Plate). Wash beads and acid elute.

Neutralization Buffer was added to wells and mixed. 100 μL/well of eluted controls/samples was added to a new non-binding plate, and 120μ/well of Biotin-XTMAB-16 working solution was added. Plates were shaken at A.T. for 60±10 mins at 450 rpm.

Wells were washed with wash buffer. Strong Block III was added to each well and plates were shaken at A.T. for 1-2 hours at 450 rpm. Coated plates were washed with wash buffer.

100 μL/well of eluted controls/samples were transferred to a TNFα coated plate. Plates were shaken at A.T. for 60±10 mins at 450 rpm and washed with wash buffer. 100 μL/well of streptavidin-HRP working solution (1:50000) was added to each well and plates were shaken at A.T. for 60±10 mins at 450 rpm.

Plates were then washed with wash buffer and TMB solution was added to wells.

Stop Solution was added to each well and plates were read on a platereader at 450 nm with a reference filter of 630 nm.

Data Analysis: The raw data (OD) were captured using a SpectraMax M2e plate reader. JMP software was used for the statistical evaluations of the cut-point factor.

### Plate Control Acceptance Criteria

Mean response (OD): NC<Plate Assay Cut-Point≤LPC<HPC.

The % CV of the average response value of NC, HPC, and LPC must be ≤25%. Plate NC is the mean of all accepted NCs. NC identified as outliers (out of the range of Mean±3*SD) were excluded from plate NC calculation.

TABLE 3
Ranges of assay responses

| | |
|---|---|
| HPC Lower Limit | 0.03 |
| LPC Lower Limit | 0.56 |
| LPC Upper Limit | 1.27 |
| NC Upper Limit | 1.92 |
| (LPC/NC) Lower Limit | 0.57 |
| (LPC/NC) Upper Limit | 0.85 |

The assay cut-point factor was 0.91. A plate-specific assay cut-point was calculated using the following formula:

$$Plate - specific assay cut - point = the mean OD value of all NC in a plate \times the assay cut - point factor$$

Two-thirds of the QC samples on every plate, at least half (50%) at each level, were required to meet the acceptance criteria.

### Sample Acceptance Criteria

The % CV of the average response value of samples was ≤25%. Samples with mean OD values≤the plate-specific assay cut-point were classified as positive. Samples with mean OD values>the plate-specific assay cut-point and % CVS 25% were classified as negative.

### Validation

A CLB assay was developed and validated for the detection of NAbs against XTMAB-16 in human serum. Validation parameters for this study included cut-point determination, sensitivity, drug tolerance, intra-assay precision and inter-assay precision, selectivity, matrix interference, specificity, stability, and robustness.

NC, HPC, and LPC were included in each run. LPC were not included for cut-point and sensitivity runs. Two sets of HPC and LPC, and four sets of NC were included in each NAb assay plate.

Assay validation included assessment of the following parameters: assay cut-point, establishment of LPC concentration, assay sensitivity and drug tolerance, intra- and inter-assay precision, system suitability (control acceptance criteria), selectivity and matrix interference, specificity, stability, and robustness.

### Determination of Cut-Point

Assay cut-point data from multiple runs analyzing drug-naïve samples were generated. At least 50 lots of individual human serum samples of healthy volunteers (50 Lots) were screened in a total of six different panels. The experimental design is outlined in Table 4.

The OD signal was normalized by plate NC mean. After outlier identification and removal, normality tests were performed on the data using JMP software. The assay cut-point factor (ACPF) was calculated based on 1% false-positive rate using the equation below based on the data distribution.

$$ACPF = “ Mean - 2.33 \times SD ” ( if data normally distributed ) or “ 1 st percentile ” ( if data not normally distributed )$$

A plate-specific screening cut-point was calculated using the following formula:

$$Plate Assay Cut - Point ( ACP ) = Mean NC OD values in a plate * A - CPF$$

TABLE 4
Experimental design and target specifications for cut-point assay

| | Experimental Design | Target Specifications |
|---|---|---|
| Cut- | At least 50 unspiked | Report data |
| Point | individual human | Individual samples demonstrating |
| | sera of healthy | a % CV >25.0% will be excluded from |
| | volunteers matrix | the final data set and will not be |
| | samples | included in the assay cut-point |
| | n: 6 minimum | calculations |
| | Replicates: 2 | At least 80% of the individual lots of |
| | Analysts: 2 minimum | sera should be included in the assay cut- |
| | Days: 3 minimum | point factor calculations. Failed lots due |
| | | to % CV >25.0% will be reanalyzed |
| | | until at least 80% of the individual lots |
| | | of sera have valid results |

### Sensitivity

Sensitivity of competitive ligand binding assay was defined by the lowest concentration at which a positive control antibody preparation consistently provided a positive signal in the assay. The experimental design is outlined in Table 5.

TABLE 5
Experimental design and target specification for sensitivity assay

| | Experimental Design | Target Specifications |
|---|---|---|
| Assay | NAb PCs (600 ng/mL) | Plate acceptance criteria is met. |
| sensitivity | prepared by spiking | Sensitivity is calculated |
| in the | NAb PC into pooled | using the linear responses |
| assay | matrix and then 1.5-fold | from a diluted NAb |
| | serially diluted, followed | PC series to interpolate the |
| | by drug removal | expected concentration of |
| | treatment. These | NAb PC equal to the ACP. |
| | samples will be measured | If the PC titer dilutions |
| | in the assay. | do not fall above the |
| | At least 7 dilutions are | assay cut-point or have |
| | suggested. At least one | dilutions that fall above, |
| | dilution point above | and subsequent |
| | the plate-specific cut-point | dilutions rise below the |
| | will be included in the | assay cut-point, the PC titer |
| | curve fitting for assay | for that curve is indeterminate. |
| | sensitivity evaluation. | The mean concentration from |
| | Replicates: 2 | all acceptable assays will be |
| | n: 6 minimum | used to describe the relative |
| | Analysts: 2 minimum | sensitivity of the assay. |
| | Days: 3 minimum | |

The LPC concentration was defined considering the sensitivity values obtained during validation.

### Determination of Drug Tolerance

XTMAB-16 was prepared in the pNHS at 2× the desired concentration of the sample. Antibody samples were prepared in the pNHS at 2× levels. Each 2× sample was combined 1:1 with each 2× drug sample. The samples were incubated for at least 1 hour at room temperature with agitation prior to analysis. Samples were frozen prior to analysis. The mean response of the PC samples preincubated with drug were compared to the response of matching PC samples. The experimental design is outlined in Table 6.

TABLE 6
Experimental design and target specifications for drug tolerance assay

| Experimental Design | Target Specifications |
|---|---|
| HPC, LPC, and NC with different | Plate acceptance criteria is met. |
| concentration levels of XTMAB-16 | The obtained results will be |
| (200, 100, 50, 25, 12.5. and 0 μg/ | used to evaluate the effect of |
| mL). | XTMAB-16. |
| n: 1 minimum | |
| Replicates: 2 | |
| Runs: 1 minimum | |

### Intra-Assay and Inter-Assay Precision

Intra-assay precision was evaluated by analyzing multiple samples of each run acceptance control level in one validation run. Intra-assay precision was calculated using NC, LPC, and HPC responses. Positive control preparations were made from six aliquots each of LPC and HPC and analyzed at 2 replicates each. The NC was analyzed from 6 aliquots and analyzed at 2 replicates each. The experimental design is outlined in Table 7.

TABLE 7
Experimental design and target specifications for intra-assay precision

| | Experimental Design | Target Specifications |
|---|---|---|
| Intra- | NC, LPC, HPC | NC |
| assay | n: 6 each for NC, LPC, | The NC responses will be reported, |
| precision | and HPC | but no intra-assay precision |
| | Replicates: 2 | acceptance criteria will be applied. |
| | Runs: 1 minimum | LPC and HPC |
| | Analysts: 1 minimum | CV ≤25.0% between replicates |
| | Days: 1 minimum | HPC signal < LPC signal ≤ ACP |

Inter-assay precision was evaluated by analyzing samples of each run acceptance control level across all applicably accepted validation runs. Inter-assay precision was calculated using the LPC/NC ratio. The experimental design is outlined in Table 8.

TABLE 8
Experimental design and target specifications for inter-assay precision

| Validation | | |
|---|---|---|
| Items | Experimental Design | Target Specifications |
| Inter- | NC, LPC, HPC | NC |
| assay | All NC, LPC, HPC in accepted | The NC responses will be |
| precision | validation runs. In addition to | reported, but no intra-assay |
| | inter-assay % CV from precision | precision acceptance criteria |
| | runs, overall statistics for PCs | will be applied. |
| | from all validation runs will be | LPC and HPC |
| | presented. | The CV of LPC/NC ratio, |
| | Runs: 6 minimum | HPC/NC ratio must be |
| | Days: 3 minimum | ≤25.0% across runs (outlier |
| | | criteria may be applied) |

### Selectivity and Matrix Interference

The selectivity was evaluated by spiking NAb LPC to at least 20 individual sera of sarcoidosis patients. NAbs spiked and unspiked matrix samples were analyzed in comparison to the ACP. Once prepared, the samples were preincubated for at least 1 hour at room temperature with agitation, and then frozen at −70° C. (−80° C. to −60° C.) prior to analysis. The experimental design is outlined in Table 9.

TABLE 9
Experimental design and target specifications for matrix selectivity assays

| Experimental Design | Target Specifications |
|---|---|
| Minimum of 20 individual | The method selectivity is considered acceptable if at least |
| samples of sarcoidosis | 80% of the unspiked samples screen negative, and at least |
| patients unspiked (blank) | 80% of the spiked samples screen positive (above the cut- |
| and spiked at LPC level. | point). |
| n: minimum 20 samples | Samples are positive only if the criteria below are met: |
| (LPC spiked and unspiked) | Assay: The mean OD Signal ≤ACP. |
| Replicates: 2 | Precision (expressed as CV): CV ≤25.0% between |
| Runs: 1 minimum | replicated |
| | Samples are negative only if the criteria below are met: |
| | Assay: The mean OD Signal > ACP. If the above criteria |
| | are not met, the experiment may be repeated with a larger |
| | number of samples and/or different level(s) of NAb, while |
| | considering relative sensitivity of assay. |

The selectivity assessment included hemolyzed and Lipemia matrix assessment. A minimum of 6 sets of plate controls at NC and LPC were assessed unspiked and spiked with interferences (hemolyzed whole blood and lipids). The experimental design is outlined in Table 10.

TABLE 10
Experimental design and target specifications for interference assays

| Parameter | Experimental Design | Target Specifications |
|---|---|---|
| Hemolysis | Minimum of 6 sets of NC, | Plate acceptance criteria is met |
| interference | LPC, and HPC unspiked and | The hemolyzed interference is considered |
| | spiked with 2% hemolyzed | acceptable if 80% of the unspiked samples |
| | whole blood | screen negative, and at least 80% of the |
| | n: minimum 6 | NAb PC spiked samples screen positive. |
| | Replicates: 2 | |
| | Runs: 1 minimum | |
| Lipemia | Minimum of 6 sets of NC, | Plate acceptance criteria is met |
| interference | LPC, and HPC unspiked and | The lipemic interference is considered |
| | spiked with 300mg/dL lipids. | acceptable if 80% of the unspiked samples |
| | n: 6 minimum | screen negative, and at least 80% of the |
| | Replicates: 2 | NAb PC spiked samples screen positive. |
| | Runs: I minimum | |

Specificity refers to the ability of detecting only the NAbs, but not other components in the matrix. In this assay, the tolerance of target (TNFα) interference was determined. Various target concentrations covering the anticipated target levels in the samples were spiked into matrix samples at HPC, LPC, and NC levels. The experimental design is outlined in Table 11.

TABLE 11
Experimental design and target specifications for specificity assays

| Experimental Design | Target Specifications |
|---|---|
| HPC, LPC, and NC with different | Plate acceptance criteria is met. |
| concentration levels of target: TNFα | The obtained results will be used |
| (1, 0.5, 0.25 and 0 μg/mL) | to evaluate the effect of TNFα. |
| n: 1 minimum | |
| Replicates: 2 | |
| Runs: 1 minimum | |

### Stability Test

Combined stressed samples with benchtop (24±6 hours or 8±2 hours) and freeze/thaw (6 or 3 cycles from −70° C.±10° C. to A.T.) were tested. The samples were prepared and stored at −70° C.±10° C. overnight (at least 12 hours) before being thawed at A.T. for the first cycle. For the subsequent freeze/thaw cycles, samples were frozen for at least 12 hours under the same conditions and then thawed. Samples were left at the benchtop for 2±1 hours before refreezing. The experimental design is outlined in Table 12.

TABLE 12
Experimental design and target specifications for benchtop
freeze/thaw stability assays

| | Experimental | |
|---|---|---|
| Parameter | Design | Target Specification |
| Benchtop | NC, LPC, | % CV ≤25.0% between replicates |
| freeze/thaw | and HPC | for LPC and HPC |
| stability | n: ≥3 | OD response: NC > ACP ≥ LPC > |
| | | HPC |
| | | At least 66.7% stability samples at |
| | | each level must meet above |
| | | acceptance criteria. |

### Evaluation of Assay Robustness

The robustness test included incubation time range. Incubation times are shown in Table 13. Three sets of HPC, LPC, and NC were applied for the robustness assay. Only qualified robustness parameters were used in sample analysis. Acceptance criteria were as follows:

$$NC , LPC , and HPC : \% CV \leq 25 \% for each replicate$$

$$The mean LPC signal and mean HPC signal must be \leq the assay cut - point calculated for the corresponding plate ,$$

$$the mean NC signal > plate assay cut - point .$$

All robustness samples at each level must be above acceptance criteria.

TABLE 13
Incubation times for each incubation step

| | Reference | Min | Max |
|---|---|---|---|
| | Incubation | Incubation | Incubation |
| Incubation Step | Time (minutes) | Time (minutes) | Time (minutes) |
| TNFα Coat Incubation | 16-48 hours | 16 hours | 48 hours |
| Acid disassociation | 30 ± 5 minutes | 25 minutes | 35 minutes |
| Biotin-XTMAB-16 | 60 ± 10 minutes | 50 minutes | 70 minutes |
| incubation | | | |
| with acid treated samples | | | |
| Streptavidin-coated beads | 60 ± 10 minutes | 50 minutes | 70 minutes |
| incubation | | | |
| Sample/drug incubation | 60 ± 10 minutes | 50 minutes | 70 minutes |
| Sample/Biotin-XTMAB- | 60 ± 10 minutes | 50 minutes | 70 minutes |
| 16 incubation | | | |
| Anti-SA HRP conjugate | 60 ± 10 minutes | 50 minutes | 70 minutes |
| incubation | | | |

All minimum and maximum incubation times listed above are target times; the actual minimum and maximum times evaluated may be adjusted.

Results from a minimum of 12 acceptable validation runs were used to determine assay control acceptance criteria to be applied to the assay controls in sample analysis.

$$Lower limit for HPC = overall mean response - 3 \times SD$$

$$Acceptance range for LPC = overall mean response \pm 3 \times SD$$

$$Upper limit for NC = overall mean response + 3 \times SD$$

Acceptance ranges for the ratio of the LPC/NC responses were determined post-validation and will not be applied to validation runs. These acceptance ranges were set to achieve a 1% failure rate.

The LPC/NC ratio is based on mean responses, where

$$LPC / NC Ratio Acceptance Range = Ratio Mean \pm 2.33 \times SD$$

An assay control table for all acceptable validation runs, as well as the LPC/NC ratio acceptance range table, will be included in the validation report.

### Determination of an Assay Cut-Point According to the Present Technology

Fifty individual human serum samples were screened six times by two analysts to calculate an assay cut-point factor. For each run, the mean OD signal of each individual serum sample was normalized by dividing the sample mean OD by the plate negative control mean OD. The normalized OD was then log-transformed.

No sample's % CV was higher than 25.0% and all 300 individual human serum values were used for the screening cut-point analysis.

Box-plot analysis was performed on all 300 normalized values. The formulas are shown in Table 14. Five (5) analytical outliers were identified.

TABLE 14
Formulas for identifying outliers

| Analytical Outlier | |
|---|---|
| Q1 (25% quartile) | −0.0129 |
| Q3 (75% quartile) | 0.0014 |
| Q3-Q1 | 0.0143 |
| High end (Q3 + 3*(Q3 − Q1)) | 0.0443 |
| Low end (Q1 − 3*(Q3 − Q1)) | −0.0558 |

### Identification of Biological Outliers

After analytical outliers were identified and excluded from analysis, the normalized and log-transformed data for each individual serum sample (295 values) were averaged across the 6 determinations, if available. Box-plot analysis was performed on the 295 averaged normalized values. The formulae for identifying biological outliers is provided in Table 15. No biological outliers were identified.

TABLE 15
Formulas for identifying biological outliers

| Biological Outlier | |
|---|---|
| Q1 (25% quartile) | −0.0099 |
| Q3 (75% quartile) | −0.0023 |
| Q3-Q1 | 0.0076 |
| High end (Q3 + 3*(Q3 − Q1)) | 0.0205 |
| Low end (Q1 − 3*(Q3 − Q1)) | −0.0327 |

### Screening Data Normality Test

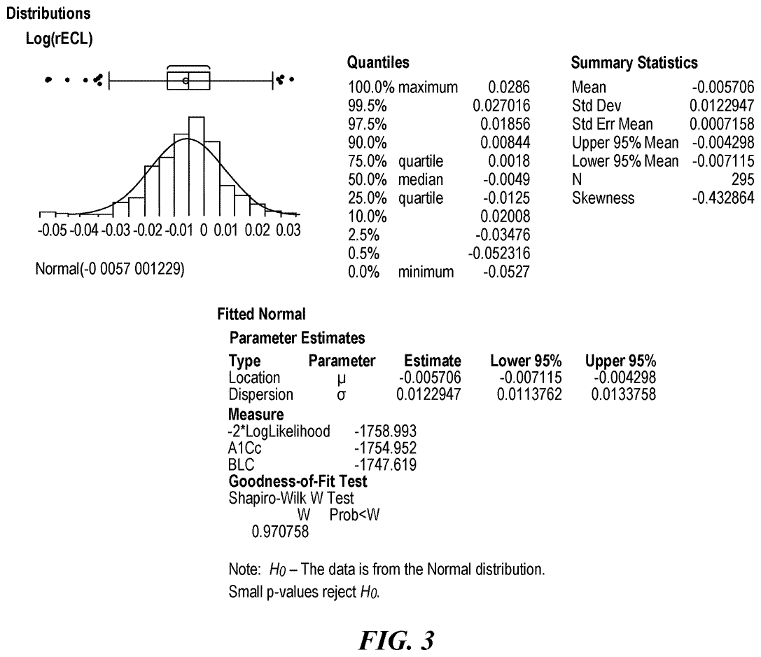

The normality of 295 log-transformed normalized values was tested by JMP 14.0 software. FIG. 3 showed that the data is not normally distributed by the Shapiro-Wilk Test. Therefore, a non-parametric approach was used to calculate a cut-point.

TABLE 16
Calculations for non-parametric
assay cut-point factors
Log(rOD) distribution
(Not Normally Distributed)
S-CPF Calculation

| | |
|---|---|
| Log(rOD, 0.99) | −0.0400 |
| S-CPF | 0.91 |

### Formulas:

$$Log ( Λ CPF , 1 \% false positive ) = 1 \% percentile of Log ( rOD ) values$$

$$Plate Assay Cut - Point ( ACP ) = Mean NC OD values in a plate * ACPF$$

### Summary

No % CV failure was identified, and all 300 individual human serum values were used for the assay cut-point analysis. Five (5) analytical outliers and no biological outliers were identified during the assay cut-point determination. The final assay ACPF were determined as 0.91, using a non-parametric approach based on 295 log-transformed normalized data values.

An example of data analyzed according to the present technology is shown in Tables 17-20, for example. Table 17 shows raw data from one run of samples that were tested according to the methods of the present technology. Table 18 shows log-transformed OD data from 6 screenings of the samples that was used to identify analytical outliers. Table 19 shows the OD data that was used to identify biological outliers after analytical outliers were removed (no biological outliers were identified). Table 20 shows the data that was used to identify the cut-point after all outliers were removed.

TABLE 17
Raw data

| Rep 1 | Rep2 | Mean | % CV | Plate NC | OD Ratio |
|---|---|---|---|---|---|
| 1.4054 | 1.4188 | 1.4121 | 0.7 | 1.4687 | 0.9615 |
| 1.3957 | 1.3596 | 1.3777 | 1.9 | | 0.9380 |
| 1.3584 | 1.3716 | 1.3650 | 0.7 | | 0.9294 |
| 1.3528 | 1.3522 | 1.3525 | 0.0 | | 0.9209 |
| 1.4321 | 1.3759 | 1.4040 | 2.8 | | 0.9559 |
| 1.4396 | 1.4136 | 1.4266 | 1.3 | | 0.9713 |
| 1.4512 | 1.4323 | 1.4418 | 0.9 | | 0.9817 |
| 1.4515 | 1.4428 | 1.4472 | 0.4 | | 0.9854 |
| 1.4155 | 1.4039 | 1.4097 | 0.6 | | 0.9598 |
| 1.4069 | 1.393 | 1.4000 | 0.7 | | 0.9532 |
| 1.4128 | 1.4164 | 1.4146 | 0.2 | | 0.9632 |
| 1.391 | 1.4129 | 1.4020 | 1.1 | | 0.9546 |
| 1.4182 | 1.4075 | 1.4129 | 0.5 | | 0.9620 |
| 1.4061 | 1.3978 | 1.4020 | 0.4 | | 0.9546 |
| 1.3775 | 1.3745 | 1.3760 | 0.2 | | 0.9369 |
| 1.449 | 1.4306 | 1.4398 | 0.9 | | 0.9803 |
| 1.3751 | 1.4128 | 1.3940 | 1.9 | | 0.9491 |
| 1.3851 | 1.4074 | 1.3963 | 1.1 | | 0.9507 |
| 1.4256 | 1.4293 | 1.4275 | 0.2 | | 0.9719 |
| 1.4064 | 1.4246 | 1.4155 | 0.9 | | 0.9638 |
| 1.4293 | 1.4322 | 1.4308 | 0.1 | | 0.9742 |
| 1.3578 | 1.3535 | 1.3557 | 0.2 | | 0.9231 |
| 1.436 | 1.4327 | 1.4344 | 0.2 | | 0.9766 |
| 1.4722 | 1.4492 | 1.4607 | 1.1 | | 0.9946 |
| 1.3534 | 1.4007 | 1.3771 | 2.4 | | 0.9376 |
| 1.3941 | 1.4251 | 1.4096 | 1.6 | | 0.9598 |
| 1.4258 | 1.42 | 1.4229 | 0.3 | | 0.9688 |
| 1.3501 | 1.3994 | 1.3748 | 2.5 | | 0.9361 |
| 1.439 | 1.4404 | 1.4397 | 0.1 | | 0.9803 |
| 1.3811 | 1.4222 | 1.4017 | 2.1 | | 0.9544 |
| 1.4444 | 1.4407 | 1.4426 | 0.2 | | 0.9822 |
| 1.4678 | 1.4518 | 1.4598 | 0.8 | | 0.9939 |

*Each row represents an individual sample

TABLE 18
Log-transformed OD data

| Screening 1 | Screening 2 | Screening 3 | Screening 4 | Screening 5 | Screening 6 |
|---|---|---|---|---|---|
| −0.0171 | −0.0026 | −0.0188 | −0.0229 | 0.0058 | −0.0098 |
| −0.0278 | −0.0725 | 0.0154 | 0.0037 | 0.0173 | −0.0097 |
| −0.0318 | 0.0023 | 0.0039 | −0.0082 | 0.0102 | −0.0129 |
| −0.0358 | −0.0724 | 0.0086 | −0.0396 | 0.0092 | −0.0092 |
| −0.0196 | −0.0108 | 0.0063 | −0.0167 | 0.0251 | −0.0099 |
| −0.0126 | −0.0013 | −0.0028 | −0.0121 | 0.0190 | −0.0105 |
| −0.0080 | 0.0048 | 0.0181 | −0.0024 | 0.0240 | 0.0032 |
| −0.0064 | −0.0063 | 0.0050 | −0.1006 | 0.0286 | −0.0067 |
| −0.0178 | 0.0025 | −0.0232 | −0.0135 | −0.0165 | −0.0030 |
| −0.0208 | 0.0109 | 0.0033 | −0.0015 | −0.0088 | 0.0021 |
| −0.0163 | 0.0075 | 0.0020 | 0.0033 | −0.0071 | −0.0018 |
| −0.0202 | 0.0003 | 0.0019 | −0.0034 | 0.0106 | −0.0022 |
| −0.0168 | 0.0101 | 0.0045 | 0.0028 | 0.0057 | 0.0085 |
| −0.0202 | 0.0125 | −0.0146 | −0.0127 | 0.0039 | 0.0040 |
| −0.0283 | −0.0015 | −0.0070 | −0.0070 | −0.0126 | 0.0083 |
| −0.0086 | 0.0044 | −0.0064 | −0.0166 | −0.0160 | −0.0013 |
| −0.0227 | −0.0063 | −0.0309 | −0.0159 | −0.0254 | −0.0016 |
| −0.0220 | 0.0057 | −0.0044 | 0.0053 | −0.0027 | 0.0007 |
| −0.0124 | −0.0066 | −0.0013 | 0.0023 | −0.0010 | 0.0047 |
| −0.0160 | 0.0016 | 0.0109 | −0.0081 | −0.0041 | −0.0031 |
| −0.0114 | 0.0044 | 0.0140 | −0.0004 | 0.0093 | −0.0007 |
| −0.0348 | −0.2314 | −0.0071 | −0.0089 | 0.0089 | 0.0012 |
| −0.0103 | −0.0037 | −0.0049 | −0.0017 | 0.0036 | −0.0072 |
| −0.0024 | 0.0124 | 0.0009 | −0.0049 | 0.0129 | −0.0056 |
| −0.0280 | −0.0153 | 0.0046 | −0.0243 | −0.0074 | −0.0129 |
| −0.0178 | −0.0015 | 0.0221 | −0.0104 | −0.0042 | −0.0156 |
| −0.0138 | −0.0037 | 0.0183 | −0.0105 | −0.0046 | −0.0007 |
| −0.0287 | −0.0350 | 0.0141 | −0.0139 | −0.0075 | −0.0179 |
| −0.0086 | −0.0018 | 0.0148 | −0.0033 | −0.0519 | −0.0101 |
| −0.0203 | −0.0018 | 0.0153 | −0.0113 | −0.0192 | −0.0101 |
| −0.0078 | 0.0069 | 0.0144 | −0.0173 | −0.0185 | −0.0013 |
| −0.0027 | 0.0156 | 0.0216 | −0.0014 | 0.0038 | −0.0036 |
| −0.0200 | −0.0097 | −0.0101 | −0.0088 | −0.0164 | −0.0018 |
| −0.0049 | −0.0027 | 0.0003 | 0.0253 | −0.0032 | 0.0038 |
| −0.0122 | −0.0057 | −0.0017 | 0.0021 | −0.0023 | −0.0003 |
| −0.0120 | −0.0075 | 0.0013 | −0.0008 | 0.0008 | −0.0123 |
| −0.0085 | −0.0158 | −0.0105 | −0.0010 | −0.0106 | −0.0146 |
| −0.0033 | −0.0037 | −0.0023 | 0.0074 | 0.0032 | −0.0005 |
| 0.0007 | −0.0067 | 0.0067 | 0.0044 | −0.0089 | −0.0116 |
| 0.0036 | −0.0113 | 0.0038 | −0.0064 | −0.0027 | −0.0112 |
| −0.0525 | −0.0094 | −0.0175 | −0.0171 | −0.0567 | −0.0114 |
| −0.0067 | −0.0094 | −0.0070 | −0.0005 | −0.0202 | 0.0013 |
| −0.0162 | −0.0061 | 0.0026 | −0.0062 | −0.0253 | 0.0006 |
| −0.0168 | −0.0175 | −0.0013 | −0.0069 | −0.0185 | −0.0045 |
| −0.0131 | −0.0084 | 0.0043 | −0.0071 | −0.0161 | −0.0008 |
| −0.0032 | −0.0012 | 0.0013 | −0.0027 | −0.0096 | −0.0002 |
| −0.0067 | −0.0141 | 0.0081 | 0.0081 | −0.0018 | 0.0003 |
| −0.0137 | −0.0207 | −0.0035 | 0.0040 | −0.0070 | −0.0276 |
| −0.0361 | −0.0194 | −0.0121 | −0.0263 | −0.0458 | −0.0129 |

*Each row represents an individual sample

TABLE 19
Log-transformed OD data with outliers removed

| Screening 1 | Screening 2 | Screening 3 | Screening 4 | Screening 5 | Screening 6 | Mean |
|---|---|---|---|---|---|---|
| −0.0171 | −0.0026 | −0.0188 | −0.0229 | 0.0058 | −0.0098 | −0.0109 |
| −0.0278 | NA | 0.0154 | 0.0037 | 0.0173 | −0.0097 | −0.0002 |
| −0.0318 | 0.0023 | 0.0039 | −0.0082 | 0.0102 | −0.0129 | −0.0061 |
| −0.0358 | NA | 0.0086 | −0.0396 | 0.0092 | −0.0092 | −0.0133 |
| −0.0196 | −0.0108 | 0.0063 | −0.0167 | 0.0251 | −0.0099 | −0.0043 |
| −0.0126 | −0.0013 | −0.0028 | −0.0121 | 0.0190 | −0.0105 | −0.0034 |
| −0.0080 | 0.0048 | 0.0181 | −0.0024 | 0.0240 | 0.0032 | 0.0066 |
| −0.0064 | −0.0063 | 0.0050 | NA | 0.0286 | −0.0067 | 0.0029 |
| −0.0178 | 0.0025 | −0.0232 | −0.0135 | −0.0165 | −0.0030 | −0.0119 |
| −0.0208 | 0.0109 | 0.0033 | −0.0015 | −0.0088 | 0.0021 | −0.0025 |
| −0.0163 | 0.0075 | 0.0020 | 0.0033 | −0.0071 | −0.0018 | −0.0021 |
| −0.0202 | 0.0003 | 0.0019 | −0.0034 | 0.0106 | −0.0022 | −0.0022 |
| −0.0168 | 0.0101 | 0.0045 | 0.0028 | 0.0057 | 0.0085 | 0.0025 |
| −0.0202 | 0.0125 | −0.0146 | −0.0127 | 0.0039 | 0.0040 | −0.0045 |
| −0.0283 | −0.0015 | −0.0070 | −0.0070 | −0.0126 | 0.0083 | −0.0080 |
| −0.0086 | 0.0044 | −0.0064 | −0.0166 | −0.0160 | −0.0013 | −0.0074 |
| −0.0227 | −0.0063 | −0.0309 | −0.0159 | −0.0254 | −0.0016 | −0.0171 |
| −0.0220 | 0.0057 | −0.0044 | 0.0053 | −0.0027 | 0.0007 | −0.0029 |
| −0.0124 | −0.0066 | −0.0013 | 0.0023 | −0.0010 | 0.0047 | −0.0024 |
| −0.0160 | 0.0016 | 0.0109 | −0.0081 | −0.0041 | −0.0031 | −0.0031 |
| −0.0114 | 0.0044 | 0.0140 | −0.0004 | 0.0093 | −0.0007 | 0.0025 |
| −0.0348 | NA | −0.0071 | −0.0089 | 0.0089 | 0.0012 | −0.0081 |
| −0.0103 | −0.0037 | −0.0049 | −0.0017 | 0.0036 | −0.0072 | −0.0040 |
| −0.0024 | 0.0124 | 0.0009 | −0.0049 | 0.0129 | −0.0056 | 0.0022 |
| −0.0280 | −0.0153 | 0.0046 | −0.0243 | −0.0074 | −0.0129 | −0.0139 |
| −0.0178 | −0.0015 | 0.0221 | −0.0104 | −0.0042 | −0.0156 | −0.0046 |
| −0.0138 | −0.0037 | 0.0183 | −0.0105 | −0.0046 | −0.0007 | −0.0025 |
| −0.0287 | −0.0350 | 0.0141 | −0.0139 | −0.0075 | −0.0179 | −0.0148 |
| −0.0086 | −0.0018 | 0.0148 | −0.0033 | −0.0519 | −0.0101 | −0.0102 |
| −0.0203 | −0.0018 | 0.0153 | −0.0113 | −0.0192 | −0.0101 | −0.0079 |
| −0.0078 | 0.0069 | 0.0144 | −0.0173 | −0.0185 | −0.0013 | −0.0039 |
| −0.0027 | 0.0156 | 0.0216 | −0.0014 | 0.0038 | −0.0036 | 0.0055 |
| −0.0200 | −0.0097 | −0.0101 | −0.0088 | −0.0164 | −0.0018 | −0.0112 |
| −0.0049 | −0.0027 | 0.0003 | 0.0253 | −0.0032 | 0.0038 | 0.0031 |
| −0.0122 | −0.0057 | −0.0017 | 0.0021 | −0.0023 | −0.0003 | −0.0034 |
| −0.0120 | −0.0075 | 0.0013 | −0.0008 | 0.0008 | −0.0123 | −0.0051 |
| −0.0085 | −0.0158 | −0.0105 | −0.0010 | −0.0106 | −0.0146 | −0.0102 |
| −0.0033 | −0.0037 | −0.0023 | 0.0074 | 0.0032 | −0.0005 | 0.0001 |
| 0.0007 | −0.0067 | 0.0067 | 0.0044 | −0.0089 | −0.0116 | −0.0026 |
| 0.0036 | −0.0113 | 0.0038 | −0.0064 | −0.0027 | −0.0112 | −0.0040 |
| −0.0525 | −0.0094 | −0.0175 | −0.0171 | NA | −0.0114 | −0.0216 |
| −0.0067 | −0.0094 | −0.0070 | −0.0005 | −0.0202 | 0.0013 | −0.0071 |
| −0.0162 | −0.0061 | 0.0026 | −0.0062 | −0.0253 | 0.0006 | −0.0084 |
| −0.0168 | −0.0175 | −0.0013 | −0.0069 | −0.0185 | −0.0045 | −0.0109 |
| −0.0131 | −0.0084 | 0.0043 | −0.0071 | −0.0161 | −0.0008 | −0.0069 |
| −0.0032 | −0.0012 | 0.0013 | −0.0027 | −0.0096 | −0.0002 | −0.0026 |
| −0.0067 | −0.0141 | 0.0081 | 0.0081 | −0.0018 | 0.0003 | −0.0010 |
| −0.0137 | −0.0207 | −0.0035 | 0.0040 | −0.0070 | −0.0276 | −0.0114 |
| −0.0361 | −0.0194 | −0.0121 | −0.0263 | −0.0458 | −0.0129 | −0.0254 |

*Each row represents an individual sample

TABLE 20
Data used to identify cut-point

| Screening 1 | Screening 2 | Screening 3 | Screening 4 | Screening 5 | Screening 6 |
|---|---|---|---|---|---|
| −0.0171 | −0.0026 | −0.0188 | −0.0229 | 0.0058 | −0.0098 |
| −0.0278 | NA | 0.0154 | 0.0037 | 0.0173 | −0.0097 |
| −0.0318 | 0.0023 | 0.0039 | −0.0082 | 0.0102 | −0.0129 |
| −0.0358 | NA | 0.0086 | −0.0396 | 0.0092 | −0.0092 |
| −0.0196 | −0.0108 | 0.0063 | −0.0167 | 0.0251 | −0.0099 |
| −0.0126 | −0.0013 | −0.0028 | −0.0121 | 0.0190 | −0.0105 |
| −0.0080 | 0.0048 | 0.0181 | −0.0024 | 0.0240 | 0.0032 |
| −0.0064 | −0.0063 | 0.0050 | NA | 0.0286 | −0.0067 |
| −0.0178 | 0.0025 | −0.0232 | −0.0135 | −0.0165 | −0.0030 |
| −0.0208 | 0.0109 | 0.0033 | −0.0015 | −0.0088 | 0.0021 |
| −0.0163 | 0.0075 | 0.0020 | 0.0033 | −0.0071 | −0.0018 |
| −0.0202 | 0.0003 | 0.0019 | −0.0034 | 0.0106 | −0.0022 |
| −0.0168 | 0.0101 | 0.0045 | 0.0028 | 0.0057 | 0.0085 |
| −0.0202 | 0.0125 | −0.0146 | −0.0127 | 0.0039 | 0.0040 |
| −0.0283 | −0.0015 | −0.0070 | −0.0070 | −0.0126 | 0.0083 |
| −0.0086 | 0.0044 | −0.0064 | −0.0166 | −0.0160 | −0.0013 |
| −0.0227 | −0.0063 | −0.0309 | −0.0159 | −0.0254 | −0.0016 |
| −0.0220 | 0.0057 | −0.0044 | 0.0053 | −0.0027 | 0.0007 |
| −0.0124 | −0.0066 | −0.0013 | 0.0023 | −0.0010 | 0.0047 |
| −0.0160 | 0.0016 | 0.0109 | −0.0081 | −0.0041 | −0.0031 |
| −0.0114 | 0.0044 | 0.0140 | −0.0004 | 0.0093 | −0.0007 |
| −0.0348 | NA | −0.0071 | −0.0089 | 0.0089 | 0.0012 |
| −0.0103 | −0.0037 | −0.0049 | −0.0017 | 0.0036 | −0.0072 |
| −0.0024 | 0.0124 | 0.0009 | −0.0049 | 0.0129 | −0.0056 |
| −0.0280 | −0.0153 | 0.0046 | −0.0243 | −0.0074 | −0.0129 |
| −0.0178 | −0.0015 | 0.0221 | −0.0104 | −0.0042 | −0.0156 |
| −0.0138 | −0.0037 | 0.0183 | −0.0105 | −0.0046 | −0.0007 |
| −0.0287 | −0.0350 | 0.0141 | −0.0139 | −0.0075 | −0.0179 |
| −0.0086 | −0.0018 | 0.0148 | −0.0033 | −0.0519 | −0.0101 |
| −0.0203 | −0.0018 | 0.0153 | −0.0113 | −0.0192 | −0.0101 |
| −0.0078 | 0.0069 | 0.0144 | −0.0173 | −0.0185 | −0.0013 |
| −0.0027 | 0.0156 | 0.0216 | −0.0014 | 0.0038 | −0.0036 |
| −0.0200 | −0.0097 | −0.0101 | −0.0088 | −0.0164 | −0.0018 |
| −0.0049 | −0.0027 | 0.0003 | 0.0253 | −0.0032 | 0.0038 |
| −0.0122 | −0.0057 | −0.0017 | 0.0021 | −0.0023 | −0.0003 |
| −0.0120 | −0.0075 | 0.0013 | −0.0008 | 0.0008 | −0.0123 |
| −0.0085 | −0.0158 | −0.0105 | −0.0010 | −0.0106 | −0.0146 |
| −0.0033 | −0.0037 | −0.0023 | 0.0074 | 0.0032 | −0.0005 |
| 0.0007 | −0.0067 | 0.0067 | 0.0044 | −0.0089 | −0.0116 |
| 0.0036 | −0.0113 | 0.0038 | −0.0064 | −0.0027 | −0.0112 |
| −0.0525 | −0.0094 | −0.0175 | −0.0171 | NA | −0.0114 |
| −0.0067 | −0.0094 | −0.0070 | −0.0005 | −0.0202 | 0.0013 |
| −0.0162 | −0.0061 | 0.0026 | −0.0062 | −0.0253 | 0.0006 |
| −0.0168 | −0.0175 | −0.0013 | −0.0069 | −0.0185 | −0.0045 |
| −0.0131 | −0.0084 | 0.0043 | −0.0071 | −0.0161 | −0.0008 |
| −0.0032 | −0.0012 | 0.0013 | −0.0027 | −0.0096 | −0.0002 |
| −0.0067 | −0.0141 | 0.0081 | 0.0081 | −0.0018 | 0.0003 |
| −0.0137 | −0.0207 | −0.0035 | 0.0040 | −0.0070 | −0.0276 |
| −0.0361 | −0.0194 | −0.0121 | −0.0263 | −0.0458 | −0.0129 |

*Each row represents an individual sample

### Validation Summary

### Assay Performance Characteristics

All samples and controls, two sets of LPC and HPC, and at least four sets of NC were evaluated in duplicate. The text files imported from the SpectraMax M2e reader were used as the source data for analysis. The assay cut-point was calculated using JMP 14.0 statistical software. The study-phase bioanalysis acceptance criteria for the OD ranges of HPC, LPC, and NC were calculated by the Responsible Scientist (RS). All mean duplicate (or quadruplicate, where applicable) OD values were assessed in the analysis.

### Assay Cut-Point

To determine the assay cut-point for the detection of NAb to XTMAB-16, two analysts tested 50 individual naïve human serum samples. Each sample was tested six times across 12 individual runs. The assay cut-point factor (0.91) was calculated as described in the present technology.

This cut-point factor was used to calculate a plate-specific assay cut-point for each assay run as follows: plate-specific assay cut-point=0.91*mean OD value of NC on each plate. Samples were classified as presumptive positive when their OD value≤plate-specific assay cut-point.

### Sensitivity

The assay sensitivity was evaluated 12 times. Serial 1.5-fold dilutions of the spiked samples (from 600.00 ng/mL to 52.67 ng/mL) in pNHS were analyzed. The assessment of sensitivity in human serum was based on the sample with the lowest concentration of ADA that consistently produced a result above the plate-specific assay cut-point. The assay sensitivity was determined for each sensitivity set using a point-to-point linear regression, by interpolation at the assay cut-point. The final assay sensitivity was calculated using a concentration among all the qualified curves. The results showed that the assay sensitivity of the assay to detect anti-XTMAB-16 antibodies was 102.7 ng/mL in human serum.

The sensitivity data was also used to determine the LPC of the assay. LPC for assay was calculated based on the corresponding sensitivity with a 1% false-positive rate, which is Mean (Sensitivity)+99%, df×SD (Sensitivity), where 99%, df=2.821 (df=9). The final LPC concentration for the assay was calculated to be 220.6 ng/mL (Table 21).

TABLE 21
Sensitivity Data

| | Assay | | | | | | | | |
|---|---|---|---|---|---|---|---|---|---|
| NAb | Sensitivity 1 | | | | | | | | |
| Cone. | | | | Plate | Plate | | Sensitivity 2 | | |
| (ng/mL) | OD | % CV | Condition | NC | ACP | Sensitivity | OD | % CV | Condition |
| 600.00 | 0.2604 | 1.6 | Pass | 1.4687 | 1.337 | NA | 0.3387 | 1.1 | Pass |
| 400.00 | 0.6690 | 4 | Pass | | | | 0.6620 | 1.0 | Pass |
| 266.67 | 0.8813 | 2.8 | Pass | | | | 0.9692 | 1.3 | Pass |
| 177.78 | 1.0626 | 0.1 | Pass | | | | 1.1320 | 1.1 | Pass |
| 118.52 | 1.1970 | 1.8 | Pass | | | | 1.2520 | 0.6 | Pass |
| 79.01 | 1.0306 | 1.3 | Pass | | | | 1.3164 | 0.4 | Pass |
| 52.67 | 1.2807 | 1.9 | Pass | | | | 1.3784 | 0.1 | Pass |

| | Assay | | | | | | | | |
|---|---|---|---|---|---|---|---|---|---|
| NAb | Sensitivity 2 | | | Sensitivity 3 | | | | | |
| Cone. | Plate | Plate | | | | | Plate | Plate | |
| (ng/mL) | NC | ACP | Sensitivity | OD | % CV | Condition | NC | ACP | Sensitivity |
| 600.00 | 1.4564 | 1.325 | 75.36 | 0.2918 | 3.2 | Pass | 1.3510 | 1.229 | 56.30 |
| 400.00 | | | | 0.6094 | 1.0 | Pass | | | |
| 266.67 | | | | 0.9454 | 0.5 | Pass | | | |
| 177.78 | | | | 1.0566 | 0.4 | Pass | | | |
| 118.52 | | | | 1.1944 | 1.9 | Pass | | | |
| 79.01 | | | | 1.1326 | 2.9 | Pass | | | |
| 52.67 | | | | 1.2444 | 0.4 | Pass | | | |

| | Assay | | | | | | | | |
|---|---|---|---|---|---|---|---|---|---|
| NAb | Sensitivity 4 | | | | | | | | |
| Cone. | | | | Plate | Plate | | Sensitivity 5 | | |
| (ng/mL) | OD | % CV | Condition | NC | ACP | Sensitivity | OD | % CV | Condition |
| 600.00 | 0.2635 | 0.4 | Pass | 1.3421 | 1.221 | 60.95 | 0.3024 | 1.1 | Pass |
| 400.00 | 0.6370 | 0.3 | Pass | | | | 0.6009 | 0.1 | Pass |
| 266.67 | 0.9093 | 0.8 | Pass | | | | 0.7891 | 0.5 | Pass |
| 177.78 | 1.0843 | 0.5 | Pass | | | | 0.8859 | 0.9 | Pass |
| 118.52 | 1.1730 | 2.2 | Pass | | | | 0.9203 | 0.4 | Pass |
| 79.01 | 1.1592 | 1.4 | Pass | | | | 0.9759 | 0.5 | Pass |
| 52.67 | 1.2493 | 0.8 | Pass | | | | 1.0007 | 1.8 | Pass |

| | Assay | | | | | | | | |
|---|---|---|---|---|---|---|---|---|---|
| NAb | Sensitivity 5 | | | Sensitivity 6 | | | | | |
| Cone. | Plate | Plate | | | | | Plate | Plate | |
| (ng/mL) | NC | ACP | Sensitivity | OD | % CV | Condition | NC | ACP | Sensitivity |
| 600.00 | 0.9590 | 0.873 | 189.62 | 0.2988 | 1.4 | Pass | 0.9670 | 0.880 | 126.88 |
| 400.00 | | | | 0.5553 | 0.4 | Pass | | | |
| 266.67 | | | | 0.7253 | 0.2 | Pass | | | |
| 177.78 | | | | 0.8435 | 0.1 | Pass | | | |
| 118.52 | | | | 0.8860 | 1.5 | Pass | | | |
| 79.01 | | | | 0.9118 | 1.1 | Pass | | | |
| 52.67 | | | | 0.9309 | 1.0 | Pass | | | |

| | Assay | | | | | | | | |
|---|---|---|---|---|---|---|---|---|---|
| NAb | Sensitivity 7 | | | | | | | | |
| Cone. | | | | Plate | Plate | | Sensitivity 8 | | |
| (ng/mL) | OD | % CV | Condition | NC | ACP | Sensitivity | OD | % CV | Condition |
| 600.00 | 0.3392 | 0.1 | Pass | 1.5392 | 1.401 | NA | 0.2718 | 0.2 | Pass |
| 400.00 | 0.7880 | 0.3 | Pass | | | | 0.7035 | 0.4 | Pass |
| 266.67 | 1.0809 | 0.6 | Pass | | | | 1.0677 | 2.4 | Pass |
| 177.78 | 1.2406 | 2.1 | Pass | | | | 1.2807 | 1.6 | Pass |
| 118.52 | 1.3682 | 2.2 | Pass | | | | 1.3702 | 0.4 | Pass |
| 79.01 | 1.4133 | 0.9 | Pass | | | | 1.4768 | 0.1 | Pass |
| 52.67 | 1.3692 | 0.6 | Pass | | | | 1.4983 | 3.1 | Pass |

| | Assay | | | | | | | | |
|---|---|---|---|---|---|---|---|---|---|
| NAb | Sensitivity 8 | | | Sensitivity 9 | | | | | |
| Cone. | Plate | Plate | | | | | Plate | Plate | |
| (ng/mL) | NC | ACP | Sensitivity | OD | % CV | Condition | NC | ACP | Sensitivity |
| 600.00 | 1.5802 | 1.438 | 93.39 | 0.3471 | 1.8 | Pass | 1.3061 | 1.189 | 142.51 |
| 400.00 | | | | 0.7176 | 3.0 | Pass | | | |
| 266.67 | | | | 0.9649 | 0.5 | Pass | | | |
| 177.78 | | | | 1.1230 | 0.7 | Pass | | | |
| 118.52 | | | | 1.2339 | 1.6 | Pass | | | |
| 79.01 | | | | 1.2570 | 0.6 | Pass | | | |
| 52.67 | | | | 1.2992 | 2.4 | Pass | | | |

### Selectivity

Selectivity is the ability of the assay to detect anti-XTMAB-16 NAb in the presence of other matrix components. NAb at the LPC level (220.6 ng/mL) was spiked into 20 lots of individual diseased human serum. One unspiked sample out of 20 samples tested positive. All 20 NAb spiked samples at the LPC level were confirmed as positive. These results meet the acceptance criteria for selectivity (Table 22).

TABLE 22
Exemplary Run Selectivity

| | | Plate | Plate | | Sample | LPC Spiked | | | | Sample |
|---|---|---|---|---|---|---|---|---|---|---|
| OD mean | % CV | NC | ACP | Condition | Condition | Individuals | OD mean | % CV | Condition | Condition |
| 1.2268 | 4.2 | 1.2338 | 1.123 | >ACP | Negative | LPC-Indi 01 | 0.7528 | 2.0 | <ACP | Positive |
| 1.2228 | 3.5 | | | >ACP | Negative | LPC-Indi 02 | 0.7745 | 2.5 | <ACP | Positive |
| 1.2613 | 2.5 | | | >ACP | Negative | LPC-Indi 03 | 0.8372 | 2.0 | <ACP | Positive |
| 1.2767 | 0.8 | | | >ACP | Negative | LPC-Indi 04 | 0.8487 | 0.1 | <ACP | Positive |
| 1.2374 | 2.1 | | | >ACP | Negative | LPC-Indi 05 | 0.8643 | 2.4 | <ACP | Positive |
| 1.2552 | 2.9 | | | >ACP | Negative | LPC-Indi 06 | 0.8178 | 0.7 | <ACP | Positive |
| 1.2516 | 0.9 | | | >ACP | Negative | LPC-Indi 07 | 0.8551 | 0.9 | <ACP | Positive |
| 1.1529 | 1.0 | | | >ACP | Negative | LPC-Indi 08 | 0.8557 | 2.9 | <ACP | Positive |
| 1.1208 | 3.0 | | | <ACP | Positive | LPC-Indi 09 | 0.7180 | 3.6 | <ACP | Positive |
| 1.2044 | 2.8 | | | >ACP | Negative | LPC-Indi 10 | 0.8229 | 0.8 | <ACP | Positive |
| 1.2258 | 0.8 | | | >ACP | Negative | LPC-Indi 11 | 0.8200 | 1.8 | <ACP | Positive |
| 1.2111 | 1.5 | | | >ACP | Negative | LPC-Indi 12 | 0.8056 | 2.8 | <ACP | Positive |
| 1.2248 | 1.4 | | | >ACP | Negative | LPC-Indi 13 | 0.8889 | 1.6 | <ACP | Positive |
| 1.2372 | 0.2 | | | >ACP | Negative | LPC-Indi 14 | 0.8669 | 1.1 | <ACP | Positive |
| 1.2021 | 0.2 | | | >ACP | Negative | LPC-Indi 15 | 0.8745 | 0.2 | <ACP | Positive |
| 1.1847 | 0.9 | | | >ACP | Negative | LPC-Indi 16 | 0.8265 | 0.2 | <ACP | Positive |
| 1.1679 | 0.0 | | | >ACP | Negative | LPC-Indi 17 | 0.8572 | 0.7 | <ACP | Positive |
| 1.2208 | 4.1 | | | >ACP | Negative | LPC-Indi 18 | 0.8451 | 3.2 | <ACP | Positive |
| 1.2226 | 0.3 | | | >ACP | Negative | LPC-Indi 19 | 0.8601 | 3.1 | <ACP | Positive |
| 1.2397 | 0.1 | | | >ACP | Negative | LPC-Indi 20 | 0.8348 | 1.8 | <ACP | Positive |

*each row represents an individual unspiked sample

Matrix interference was evaluated in 300 mg/dL lipemia or 2% hemolyzed samples. Six pooled serum samples with 300 mg/dL triglycerides were assessed unspiked and spiked at LPC level (220.6 ng/mL) and HPC level (600 ng/mL). As shown in FIG. 7, all the unspiked samples were confirmed negative, and all the LPC and HPC spiked samples were confirmed positive. These results meet the acceptance criteria, indicating that 300 mg/dL lipemia matrix do not interfere with the assay results.

Six pooled serum samples with 2% hemolyzed serum were assessed unspiked and spiked at LPC level (220.6 ng/mL) and HPC level (600 ng/mL). As shown in Table 23, all the unspiked samples were confirmed negative, and all the LPC and HPC spiked samples were confirmed positive. These results meet the acceptance criteria, indicating that 2% hemolyzed matrix do not interfere with the assay results.

TABLE 23
Matrix Interference Data

| Example Run: Lipemia Matrix Interference | | | | | | Example Run: Hemolyzed Matrix Interference | | | | |
|---|---|---|---|---|---|---|---|---|---|---|
| | Samples | OD | | | Sample | Samples | OD | | | Sample |
| Samples | ID | mean | % CV | Condition | Condition | ID | mean | % CV | Condition | Condition |
| Unspiked | 1 | 1.4978 | 3.3 | >ACP | Negative | 1 | 1.5336 | 0.9 | >ACP | Negative |
| Samples | 2 | 1.4829 | 0.1 | >ACP | Negative | 2 | 1.5391 | 0.3 | >ACP | Negative |
| | 3 | 1.5208 | 0.4 | >ACP | Negative | 3 | 1.5387 | 0.9 | >ACP | Negative |
| | 4 | 1.5706 | 0.3 | >ACP | Negative | 4 | 1.5860 | 1.0 | >ACP | Negative |
| | 5 | 1.5369 | 1.0 | >ACP | Negative | 5 | 1.5636 | 0.3 | >ACP | Negative |
| | 6 | 1.5489 | 0.0 | >ACP | Negative | 6 | 1.5692 | 0.1 | >ACP | Negative |
| LPC | 1 | 1.0047 | 1.1 | <ACP | Positive | 1 | 1.0238 | 1.4 | <ACP | Positive |
| Spiked | 2 | 1.0258 | 2.4 | <ACP | Positive | 2 | 1.0360 | 2.6 | <ACP | Positive |
| Samples | 3 | 1.0292 | 1.2 | <ACP | Positive | 3 | 1.0258 | 2.5 | <ACP | Positive |
| | 4 | 1.0263 | 0.4 | <ACP | Positive | 4 | 1.0340 | 0.6 | <ACP | Positive |
| | 5 | 1.0516 | 0.2 | <ACP | Positive | 5 | 1.0778 | 0.9 | <ACP | Positive |
| | 6 | 1.0692 | 2.3 | <ACP | Positive | 6 | 1.0691 | 2.0 | <ACP | Positive |
| HPC | 1 | 0.2197 | 2.3 | <ACP | Positive | 1 | 0.2326 | 1.1 | <ACP | Positive |
| Spiked | 2 | 0.2269 | 1.7 | <ACP | Positive | 2 | 0.2111 | 1.9 | <ACP | Positive |
| Samples | 3 | 0.2161 | 1.5 | <ACP | Positive | 3 | 0.2266 | 0.7 | <ACP | Positive |
| | 4 | 0.2185 | 2.5 | <ACP | Positive | 4 | 0.2258 | 3.4 | <ACP | Positive |
| | 5 | 0.2349 | 10.8 | <ACP | Positive | 5 | 0.2505 | 0.3 | <ACP | Positive |
| | 6 | 0.2266 | 3.7 | <ACP | Positive | 6 | 0.2728 | 1.5 | <ACP | Positive |

| | | |
|---|---|---|
| Plate NC | 1.5428 | 1.5486 |
| CPF | 0.91 | 0.91 |
| Plate ACP | 1.404 | 1.409 |

### Drug Tolerance

The presence of drug (XTMAB-16) in the samples may interfere with the detection of anti-XTMAB-16 NAb and, hence, the ability of the assay system to correctly detect the NAb must be evaluated. Drug tolerance is defined as the highest level of drug that does not change the samples/controls from positive to negative or negative to positive.

Potential interference by the drug was evaluated using serum prepared at the NC level (0.0 ng/mL), LPC level (220.6 ng/mL), and HPC level (600.0 ng/mL). All three levels of controls were spiked with XTMAB-16 at concentrations at 0.0, 12.5, 25.0, 50.0, 100.0, and 200.0 μg/mL.

As shown in Table 24, no interference was found with the addition of up to 200 μg/mL XTMAB-16 to NC, LPC, and HPC levels.

TABLE 24
Interference Data
Example Run: Drug Tolerance

| Drug | NAb PC at NC level (0 ng/mL) | | | | NAb PC at LPC level (220.6 ng/mL) | | | | NAb PC at HPC level (600 ng/mL) | | | |
|---|---|---|---|---|---|---|---|---|---|---|---|---|
| Cone. | resulting | OD | % | | resulting | OD | % | | resulting | OD | % | |
| (μg/mL) | tube ID | mean | CV | Condition | tube ID | mean | CV | Condition | tube ID | mean | CV | Condition |
| 200.00 | 100 | 1.4147 | 0.2 | >ACP | 100 | 1.0566 | 3.5 | <ACP | 100 | 0.2612 | 2.1 | <ACP |
| 100.00 | 200 | 1.4034 | 0.4 | >ACP | 200 | 1.1017 | 1.8 | <ACP | 200 | 0.2320 | 2.7 | <ACP |
| 50.00 | 300 | 1.4700 | 1.1 | >ACP | 300 | 1.0561 | 1.7 | <ACP | 300 | 0.2267 | 0.2 | <ACP |
| 25.00 | 400 | 1.4626 | 2.4 | >ACP | 400 | 1.0613 | 0.4 | <ACP | 400 | 0.2222 | 0.8 | <ACP |
| 12.50 | 500 | 1.4840 | 1.3 | >ACP | 500 | 1.0479 | 1.7 | <ACP | 500 | 0.2216 | 0.4 | <ACP |
| 0.00 | 600 | 1.5169 | 2.1 | >ACP | 600 | 1.0572 | 1.0 | <ACP | 600 | 0.2152 | 1.6 | <ACP |
| Plate NC | 1.5343 | | | | | | | | | | | |
| CPF | 0.91 | | | | | | | | | | | |
| Plate ACP | 1.396 | | | | | | | | | | | |
| Drug | >200 | | | | >200 | | | | >200 | | | |
| Tolerance | | | | | | | | | | | | |
| Cone. | | | | | | | | | | | | |
| (ug/mL) | | | | | | | | | | | | |

### Target Tolerance

The presence of target (TNFα) in the samples may interfere with the detection of anti-XTMAB-16 NAb. Target tolerance is defined as the highest level of target that does not change the samples/controls from positive to negative or negative to positive.

Potential interference from the target, TNFα, was evaluated using serum prepared at the NC level (0.0 ng/mL), LPC level (220.6 ng/mL), and HPC level (600.0 ng/mL). Four samples of each control level were prepared and spiked with 0.0, 0.25, 0.50, or 1.0 μg/mL TNFα. The addition of 0.96 μg/mL of TNFα interfered with NAb detection in samples spiked at the NC level but no target interference was observed in samples spiked at the HPC and LPC level with up to 1 μg/mL TNFα (Table 25).

TABLE 25
Interference Data from Target
Example Run: Target Interference

| | | | | | | Target |
|---|---|---|---|---|---|---|
| | Samples | OD | | | Sample | tolerance |
| | ID | mean | % CV | Condition | Condition | (μg/mL) |
| NC- | 1 | 1.1396 | 2.7 | <ACP | Positive | 0.96 |
| Target | 2 | 1.1799 | 2.1 | >ACP | Negative | |
| | 3 | 1.1890 | 0.1 | >ACP | Negative | |
| | 4 | 1.2391 | 1.6 | >ACP | Negative | |
| LPC- | 1 | 0.7038 | 2.1 | <ACP | Positive | >1.0 |
| Target | 2 | 0.7177 | 3.3 | <ACP | Positive | |
| | 3 | 0.8447 | 2.2 | <ACP | Positive | |
| | 4 | 0.8053 | 2.6 | <ACP | Positive | |
| HPC- | 1 | 0.1412 | 4.5 | <ACP | Positive | >1.0 |
| Target | 2 | 0.1654 | 7.2 | <ACP | Positive | |
| | 3 | 0.1671 | 5.2 | <ACP | Positive | |
| | 4 | 0.1183 | 5.6 | <ACP | Positive | |

### Precision

Intra-assay precision, an evaluation of the within-run performance of the controls, was evaluated in one run using six sets of the HPC (600.0 ng/mL), LPC (220.6 ng/mL), and NC (0.0 ng/mL). The controls should meet the criteria of OD of NC>Plate-specific assay cut-point ≥LPC>HPC for the assay. The CV in intra-assay precision for the OD of HPC and LPC should also meet ≤25.0%. All plate control meets the above criteria. The results shown in Table 26, demonstrated that the intra-assay precision of the HPC, LPC, and NC were 21.6%, 5.6%, and 3.0%, respectively.

TABLE 26
Intra-Assay Precision
Example Run: Intra-Assay Precision

| | | |
|---|---|---|
| HPC | LPC | NC |

| | OD | % | OD | % | OD | % |
|---|---|---|---|---|---|---|
| Replicate | mean | CV | mean | CV | mean | CV |
| 1 | 0.2021 | 2.6 | 0.7895 | 3.6 | 1.1375 | 3.3 |
| 2 | 0.1742 | 0.4 | 0.8175 | 1.1 | 1.2196 | 4.5 |
| 3 | 0.2466 | 3.2 | 0.8776 | 1.1 | 1.2304 | 4.6 |
| 4 | 0.1709 | 3.4 | 0.8782 | 4.4 | 1.2015 | 0.4 |
| 5 | 0.2636 | 0.8 | 0.8689 | 1.0 | 1.2134 | 1.1 |
| 6 | 0.2860 | 2.5 | 0.9244 | 3.3 | 1.2394 | 0.2 |

| | | | |
|---|---|---|---|
| Mean | 0.2239 | 0.8594 | 1.2070 |
| % CV | 21.6 | 5.6 | 3.0 |

| | |
|---|---|
| Plate NC | 1.2070 |
| CPF | 0.91 |
| Plate ACP | 1.098 |

Inter-assay precision, an evaluation of the between-run performance of the controls, was evaluated based on the LPC/NC and HPC/NC across all accepted validation runs as listed in Tables 27-29. The inter-assay precision of the LPC/NC and HPC/NC were 8.5% and 24.6%, respectively, after removal of the outliers. The results shown in Tables 27-29 demonstrated that the controls met the inter-assay precision criteria.

TABLE 27
Inter-Assay Precision (Part 1)

| | | | | | | | HPC |
|---|---|---|---|---|---|---|---|
| | | | | | | | (600 |
| | | | | | LPC | HPC | ng/mL)/NC |
| | | | LPC | HPC | (220.6 | (600 | (after |
| Run | Validation | NC(OD | (220.6 | (600 | ng/mL)/ | ng/mL)/ | removal of |
| ID | Parameter | Mean) | ng/mL) | ng/mL) | NC | NC | the outliers) |
| 1 | Assay Cut Point/ | 1.4687 | | | | | 0.22 |
| | Sensitivity | | NA | 0.3943 | NA | 0.27 | 0.27 |
| 2 | Assay Cut Point/ | 1.4564 | NA | 0.3640 | NA | 0.25 | 0.25 |
| | Sensitivity | | NA | 0.3504 | NA | 0.24 | 0.24 |
| 3 | Assay Cut Point/ | 1.3510 | NA | 0.2774 | NA | 0.21 | 0.21 |
| | Sensitivity | | NA | 0.3482 | NA | 0.26 | 0.26 |
| 4 | Assay Cut Point/ | 1.3421 | NA | 0.2888 | NA | 0.22 | 0.22 |
| | Sensitivity | | NA | 0.3022 | NA | 0.23 | 0.23 |
| 5 | Assay Cut Point/ | 0.9590 | NA | 0.3146 | NA | 0.33 | 0.33 |
| | Sensitivity | | NA | 0.3218 | NA | 0.34 | 0.34 |
| 6 | Assay Cut Point/ | 0.9670 | NA | 0.3090 | NA | 0.32 | 0.32 |
| | Sensitivity | | NA | 0.3092 | NA | 0.32 | 0.32 |
| 7 | Assay Cut Point/ | 1.5392 | NA | 0.3179 | NA | 0.21 | 0.21 |
| | Sensitivity | | NA | 0.3903 | NA | 0.25 | 0.25 |
| 8 | Assay Cut Point/ | 1.5802 | NA | 0.3156 | NA | 0.20 | 0.20 |
| | Sensitivity | | NA | 0.3014 | NA | 0.19 | 0.19 |
| 9 | Assay Cut Point/ | 1.3061 | NA | 0.3433 | NA | 0.26 | 0.26 |
| | Sensitivity | | NA | 0.3664 | NA | 0.28 | 0.28 |
| 10 | Assay Cut Point/ | 1.3913 | NA | 0.2874 | NA | 0.21 | 0.21 |
| | Sensitivity | | NA | 0.2984 | NA | 0.21 | 0.21 |
| 11 | Assay Cut Point/ | 1.5605 | NA | 0.5377 | NA | 0.34 | 0.34 |
| | Sensitivity | | NA | 0.6667 | NA | 0.43 | NA |
| 12 | Assay Cut Point/ | 1.5563 | NA | 0.4874 | NA | 0.31 | 0.31 |
| | Sensitivity | | NA | 0.5974 | NA | 0.38 | NA |
| 13 | Drug Tolerance | 1.5343 | 1.0383 | 0.3575 | 0.68 | 0.23 | 0.23 |
| | | | 1.1039 | 0.3803 | 0.72 | 0.25 | 0.25 |

TABLE 28
Inter-Assay Precision (Part 2)

| | | | | | | | HPC |
|---|---|---|---|---|---|---|---|
| | | | | | | | (600 |
| | | | | | LPC | HPC | ng/mL)/NC |
| | | | LPC | HPC | (220.6 | (600 | (After |
| Run | Validation | NC(OD | (220.6 | (600 | ng/mL)/ | ng/mL)/ | removal of |
| ID | Parameter | Mean) | ng/mL) | ng/mL) | NC | NC | the outliers) |
| 14 | Matrix effect | 1.5428 | 1.0809 | 0.3461 | 0.70 | 0.22 | 0.22 |
| | (Lipemia) | | 1.0947 | 0.3801 | 0.71 | 0.25 | 0.25 |
| 15 | Matrix effect | 1.5486 | 1.0541 | 0.3286 | 0.68 | 0.21 | 0.21 |
| | (Hemolysis) | | 1.0746 | 0.3926 | 0.69 | 0.25 | 0.25 |
| 16 | Selectivity | 1.2338 | 0.8508 | 0.2173 | 0.69 | 0.18 | 0.18 |
| | | | 0.8375 | 0.2975 | 0.68 | 0.24 | 0.24 |
| 17 | Intra Assay | 1.2070 | 0.7895 | 0.2021 | 0.65 | 0.17 | 0.17 |
| | Precision | | 0.8175 | 0.1742 | 0.68 | 0.14 | 0.14 |
| | | | 0.8776 | 0.2466 | 0.73 | 0.20 | 0.20 |
| | | | 0.8782 | 0.1709 | 0.73 | 0.14 | 0.14 |
| | | | 0.8689 | 0.2636 | 0.72 | 0.22 | 0.22 |
| | | | 0.9244 | 0.2860 | 0.77 | 0.24 | 0.24 |
| 18 | Specificity (Target | 1.2556 | 0.7833 | 0.1638 | 0.62 | 0.13 | 0.13 |
| | Interference) | | 0.8857 | 0.2837 | 0.71 | 0.23 | 0.23 |
| 19 | Stability (Bench | 1.1412 | 0.7108 | 0.2602 | 0.62 | 0.23 | 0.23 |
| | Top) | | 0.7766 | 0.2589 | 0.68 | 0.23 | 0.23 |
| 20 | Robustness (Min | 1.1109 | 0.8122 | 0.2194 | 0.73 | 0.20 | 0.20 |
| | time) | | 0.8292 | 0.2516 | 0.75 | 0.23 | 0.23 |
| | | | 0.8123 | 0.2685 | 0.73 | 0.24 | 0.24 |
| 21 | Stability | 1.3686 | 0.8390 | 0.1386 | 0.61 | 0.10 | 0.10 |
| | (Freeze/Thaw) | | 1.0212 | 0.1330 | 0.75 | 0.10 | 0.10 |
| 22 | Robustness (Max | 1.2456 | 0.9377 | 0.1554 | 0.75 | 0.12 | 0.12 |
| | time) | | 1.0208 | 0.2700 | 0.82 | 0.22 | 0.22 |
| | | | 1.0738 | 0.2958 | 0.86 | 0.24 | 0.24 |

TABLE 29
Inter-Assay Precision (Part 3)

| | | | HPC (600 | | | HPC/NC |
|---|---|---|---|---|---|---|
| | | LPC | ng/mL) (After | | | (After |
| | | (220.6 | removal of the | LPC (220.6 | HPC (600 | removal of |
| | NC | ng/mL) | outliers) | ng/mL)/NC | ng/mL)/NC | the outliers) |
| Mean | 1.3565 | 0.9151 | 0.3037 | 0.7100 | 0.2338 | 0.2267 |
| S.D. | 0.2 | 0.1 | 0.1 | 0.1 | 0.1 | 0.1 |
| % CV | 13.8 | 12.8 | 29.9 | 8.5 | 27.8 | 24.6 |

### Stability

The HPC, LPC, and NC controls in pooled human serum were used to assess sample stability. Freeze/thaw (6 cycles of −70° C.±10° C. to A.T.) and benchtop (23 hours) stability were tested. The acceptance criteria for the stability samples were as follows:

$$\% CV \leq 25 \% for the LPC and HPC .$$

$$The mean OD : NC > ACP 2 : LPC > HPC$$

At least 66.7% stability samples must meet the above acceptance criteria.

The tested samples were stored at −70° C. (nominal) for at least 12 hours prior to first thaw and at least 12 hours prior to each additional thaw. Samples were at A.T. for 2±1 hours before refreezing. Three aliquots for each control (HPC, LPC, and NC) were tested after undergoing six freeze/thaw cycles on a plate with frozen PCs and NC, which were used for plate acceptance. All stability samples met the acceptance criteria (Table 30); therefore, anti-XTMAB-16 NAb in human serum are stable up to six freeze/thaw cycles from −70° C. (nominal) to A.T.

TABLE 30
Freeze/Thaw Stability

| Example Run: Bench Top Stability (23 hours) | | | | Example Run: Freeze-Thaw Stability (6 cycles) | | | |
|---|---|---|---|---|---|---|---|
| | | | Condition | | | | Condition |
| Sample | OD | | (Pass or | Sample | OD | | (Pass or |
| ID | mean | % CV | Fail) | ID | mean | % CV | Fail) |
| HPC 1 | 0.2326 | 2.7 | Pass | FT-HPC 1 | 0.3012 | 2.5 | Pass |
| HPC 2 | 0.2389 | 3 | Pass | FT-HPC2 | 0.3146 | 0.4 | Pass |
| HPC 3 | 0.2215 | 3.5 | Pass | FT-HPC 3 | 0.3084 | 0.1 | Pass |
| LPC 1 | 0.8199 | 1.6 | Pass | FT-LPC 1 | 0.9661 | 0.2 | Pass |
| LPC 2 | 0.8371 | 1.1 | Pass | FT-LPC 2 | 1.0143 | 4.4 | Pass |
| LPC 3 | 0.8119 | 1.1 | Pass | FT-LPC 3 | 1.0274 | 1.6 | Pass |
| NC 1 | 1.0706 | 4.2 | Pass | FT-NC 1 | 1.2508 | 6.1 | Pass |
| NC2 | 1.0743 | 1.1 | Pass | FT-NC2 | 1.2512 | 5.7 | Pass |
| NC 3 | 1.095 | 0.7 | Pass | FT-NC 3 | 1.2755 | 2.6 | Pass |

| | | |
|---|---|---|
| Plate NC | 1.1412 | 1.3686 |
| CPF | 0.91 | 0.91 |
| Plate ACP | 1.038 | 1.245 |

The tested samples were stored at −70° C. (nominal) overnight prior to stability testing. Three aliquots of each pre-existing frozen control (HPC, LPC, and NC) were thawed on the benchtop for 23 hours, prior to analysis with frozen PCs and NC, which were used for plate acceptance. All stability samples met the acceptance criteria (Table 30). Therefore, anti-XTMAB-16 NAb in human serum are stable up to 23 hours at A.T. (benchtop).

### Study-Phase Bioanalysis Acceptance Criteria for Controls

System suitability criteria were based on the data from all valid runs in the validation runs. The % CV criterion for intra-replicate measurements of controls and samples is ≤25%.

Acceptance criteria for NC:

$$Upper limit for NC = overall mean response + 3 \times SD and > ACP$$

The upper limit of OD value for NC is 1.92, inclusive.

Acceptance criteria for LPC:

$$Acceptance range for LPC = overall mean response \pm 3 \times SD and \leq ACP .$$

Range OD values for LPC (220.6 ng/mL XTMAB-16 NAb in pNHS) is 0.56 to 1.27, inclusive.

Acceptance criteria for HPC:

$$Lower limit for HPC = overall mean response - 3 \times SD and < LPC .$$

The lower limit of OD value for HPC (600.0 ng/mL XTMAB-16 NAb in pNHS) is 0.03, inclusive.

Acceptance criteria for LPC/INC:

$$LPC / NC Ratio Acceptance Range = Ratio Mean \pm 2.33 \times SD$$

Range OD values for LPC/NC is 0.57 to 0.85, inclusive.

### Assay Robustness

To determine assay robustness, the parameters of the assay were tested. The HPC, LPC, and NC were tested in both assay and confirmatory assay with different incubation times in two runs as per the conditions noted in Table 13.

The runs met the method acceptance criteria, and the controls were within the established ranges. As shown in Table 31, the assay demonstrated robustness at the maximum and minimum incubation times.

TABLE 31
Robustness at Minimum and Maximum Incubation Times

| | Robustness | | | | | |
|---|---|---|---|---|---|---|
| | Example Run: Min Time | | | Example Run: Max Time | | |
| Sample ID | OD mean | % CV | Condition | OD mean | % CV | Condition |
| HPC | 0.2194 | 2.2 | Pass | 0.1554 | 1.4 | Pass |
| | 0.2516 | 5.2 | Pass | 0.2700 | 4.3 | Pass |
| | 0.2685 | 2.1 | Pass | 0.2958 | 3.8 | Pass |
| LPC | 0.8122 | 0.3 | Pass | 0.9377 | 5.2 | Pass |
| | 0.8292 | 0.4 | Pass | 1.0208 | 4.6 | Pass |
| | 0.8123 | 0.7 | Pass | 1.0738 | 0.3 | Pass |
| NC | 1.0522 | 4.2 | Pass | 1.2047 | 0.4 | Pass |
| | 1.0985 | 0.3 | Pass | 1.2222 | 0.4 | Pass |
| | 1.1820 | 2.3 | Pass | 1.3099 | 1.7 | Pass |

| | | |
|---|---|---|
| Plate ACP | 1.011 | 1.133 |

| | |
|---|---|
| CPF | 0.91 |

TABLE 32
Summary of results

| Validation Parameter | Results |
|---|---|
| Assay cut-point factor | 0.91 |
| Relative sensitivity | 102.7 ng/ml |
| Selectivity (20 | 95% unspiked samples were negative |
| sarcoidosis disease | 100% spiked samples at 220.6 ng/ml (LPC) |
| individual samples) | were positive |
| Matrix interference | 100% unspiked samples were negative |
| (6 hemolyzed and 6 | 100% spiked samples at 220.6 ng/ml (LPC) |
| lipemic) | were positive |
| Drug tolerance | Up to 200 μg/mL at LPC and PC1000 |
| Assay precision | Inter % CV-HPC/NC: 24.6, LPC/NC: 8.5 |
| | Intra % CV-HPC/NC: 15.7, LPC/NC: 7.2 |
| Hook effect | No hook effect was observed at |
| | concentration up to 20 μg/mL |
| Target interference | No interference was observed at the LPC |
| | spiked samples with 1 μg/mL of TNFα |
| Stability | QCs are stable up to 24 hours at 2° C.-8° C., |
| | and 6 freeze/thaw cycles from −70° C. |

### Additional Embodiments

Clause 1. A method for detecting a NAb (neutralizing antibody) against a target antibody in a sample comprising: (i) disassociating the NAb from the target antibody by adding an acid wash to the sample; (ii) purifying the NAb in the sample; (iii) contacting the purified NAb with a detection antibody comprising an anti-TNFα antibody conjugated to a detection label; (iv) contacting the purified NAb and the detection antibody with a TNFα-coated substrate; and (v) detecting the NAb when the detection antibody binds the NAb instead of the TNFα-coated substrate, wherein the target antibody comprises an anti-TNFα chimeric human-murine monoclonal IgG1-kappa antibody comprising SEQ ID NO: 9-11 or 15-17 and SEQ ID NOs: 12-14 or 18-20.

Clause 2. The method of clause 1, further comprising detecting the detection label before detecting the NAb.

Clause 3. The method of clause 1, further comprising washing the TNFα-coated substrate before detecting the NAb.

Clause 4. The method of clause 1, wherein the acid wash comprises acetic acid.

Clause 5. The method of clause 1 or 4, wherein purifying the NAb of step (ii) comprises: (a) contacting the sample with an affinity antibody comprising anti-TNFα antibody conjugated to a first affinity label; (b) contacting the sample with a first solid phase coated with a second affinity label, wherein the first affinity label binds to the second affinity label; (c) washing the first solid phase; and (d) eluting the NAb from the first solid phase.

Clause 6. The method of clause 5, wherein the first affinity label is biotin.

Clause 7. The method of clause 5 or 6, wherein the first solid phase is a microbead.

Clause 8. The method of any one of clauses 5-7, wherein the first solid phase is a magnetic microbead.

Clause 9. The method of any one of clauses 5-8, wherein the second affinity label is streptavidin.

Clause 10. The method of any one of clauses 1-9, wherein the NAb is purified using an automated purification system.

Clause 11. The method of any one of clauses 1-9, wherein the detection label comprises a luminescent compound.

Clause 12. The method of any one of clauses 1-9, wherein the detection label comprises an enzyme configured to convert a chromogenic substrate into a pigment.

Clause 13. The method of any one of clauses 1-9, wherein the detection label is a third affinity label.

Clause 14. The method of clause 13, wherein detecting the detection label further comprises contacting the third affinity label with a fourth affinity label conjugated to a luminescent compound or enzyme configured to convert a chromogenic substrate into a pigment, wherein the third affinity label binds to the fourth affinity label.

Clause 15. The method of clause 14, wherein the third affinity label is biotin, and the fourth affinity label is streptavidin.

Clause 16. The method of clause 11 or 14, wherein detecting the detection label comprises detecting the luminescent compound.

Clause 17. The method of clause 12 or 14, wherein detecting the detection label comprises converting the chromogenic substrate into the pigment and detecting the pigment.

Clause 18. The method of clause 12 or 14, wherein detecting the detection label comprises converting the chromogenic substrate into the pigment and detecting an optical density of the sample.

Clause 19. The method of any one of clauses 12 or 14-18, wherein the enzyme is a horseradish peroxidase (HRP).

Clause 20. The method of clause 19, wherein the chromogenic substrate is 3,3′,5,5′-tetramethylbenzidine (TMB).

Clause 21. The method of any one of clauses 1-20, wherein steps (i) to (v) are performed on a negative control sample having no detectable NAb.

Clause 22. The method of any of clauses 1-21, wherein the detection antibody binding the NAb instead of the TNFα-coated substrate is detected when the detection label is present at a level below a predetermined level.

Clause 23. The method of clause 22, wherein the detection label is present at the level below the predetermined level when an optical density of the sample is above a predetermined cut-point.

Clause 24. The method of clause 23, wherein the predetermined cut-point is the level corresponding to a false-positive rate of 3% or less.

Clause 25. The method of any one of clauses 23-24, wherein the predetermined cut-point is the level corresponding to a 1% false-positive rate.

Clause 26. The method of any one of clauses 23-25, wherein the predetermined cut-point is 0.5-2.

Clause 27. The method of clause 26, wherein the predetermined cut-point is 0.91.

Clause 28. The method of any one of clauses 1-27, wherein a sensitivity of detecting the NAb is less than 500 ng/mL.

Clause 29. The method of clause 28, wherein the sensitivity of detecting the NAb is less than 250 ng/mL.

Clause 30. The method of any one of clauses 28 or 29, wherein the sensitivity of detecting the NAb is less than 150 ng/mL.

Clause 31. The method of any one of clauses 1-30, wherein a target tolerance is about 1000 ng/mL.

Clause 32. The method of any one of clauses 1-31, wherein the target antibody comprises SEQ ID NOs: 2 and 5.

Clause 33. The method of any one of clauses 1-32, wherein the target antibody comprises SEQ ID NOs: 1 and 4.

Clause 34. A method for detecting a NAb against a target antibody in a sample comprising: (i) purifying the NAb in the sample; (ii) determining whether the NAb is present in the sample by performing a competitive binding assay, wherein the NAb is present if the NAb binds the anti-TNFα antibody with a higher affinity than the affinity of the anti-TNFα antibody to TNFα; and (iii) detecting the NAb; wherein a sensitivity of the method is 250 ng/mL or less, wherein the target antibody comprises an anti-TNFα chimeric human-murine monoclonal IgG1-kappa antibody comprising SEQ ID NO: 9-11 or 15-17 and SEQ ID NOs: 12-14 or 18-20.

Clause 35. The method of clause 34, wherein before the NAb is purified, the method further comprises disassociating the NAb from the target antibody by adding an acid wash to the sample.

Clause 36. The method of clause 34 or 35, wherein the competitive binding assay comprises: (a) contacting the purified NAb with a detection antibody comprising an anti-TNFα antibody conjugated to a detection label; (b) contacting the purified NAb and the detection antibody with a TNFα-coated substrate; (c) washing the TNFα-coated substrate; and (d) detecting the detection label.

Clause 37. The method of clause 36, wherein detecting the NAb comprises determining whether the detection label is present at a level below a predetermined level.

Clause 38. The method of clause 37, wherein the detection label is present at the level below the predetermined level when an optical density is above a predetermined cut-point.

Clause 39. The method of any one of clauses 35-38, wherein the acid wash comprises acetic acid.

Clause 40. The method of any one of clauses 34-39, wherein purifying the NAb comprises: (a) contacting the sample with an affinity antibody comprising an anti-TNFα antibody conjugated to a first affinity label; (b) contacting the sample with a first solid phase coated with a second affinity label, wherein the first affinity label binds to the second affinity label; (c) washing the first solid phase; and (d) eluting the NAb from the first solid phase.

Clause 41. The method of clause 40, wherein the first affinity label is biotin.

Clause 42. The method of clause 40 or 41, wherein the first solid phase is a microbead.

Clause 43. The method of any one of clauses 40-42, wherein the first solid phase is a magnetic microbead.

Clause 44. The method of any one of clauses 40-43, wherein the second affinity label is streptavidin.

Clause 45. The method of any one of clauses 34-44, wherein the NAb is purified using an automated purification system.

Clause 46. The method of any one of clauses 36-45, wherein the detection label comprises a luminescent compound.

Clause 47. The method of any one of clauses 36-45, wherein the detection label comprises an enzyme configured to convert a chromogenic substrate into a pigment.

Clause 48. The method of any one of clauses 36-45, wherein the detection label is a third affinity label.

Clause 49. The method of clause 48, wherein detecting the detection label further comprises contacting the third affinity label with a fourth affinity label conjugated to a luminescent compound or enzyme configured to convert a chromogenic substrate into a pigment, wherein the third affinity label binds to the fourth affinity label.

Clause 50. The method of clause 49, wherein the third affinity label is biotin, and the fourth affinity label is streptavidin.

Clause 51. The method of clause 46 or 49, wherein detecting the detection label comprises detecting the luminescent compound.

Clause 52. The method of clause 47 or 49, wherein detecting the detection label comprises converting the chromogenic substrate into the pigment and detecting the pigment.

Clause 53. The method of clause 47 or 49, wherein detecting the detection label comprises converting the chromogenic substrate into the pigment and detecting an optical density of the sample.

Clause 54. The method of any one of clauses 47 or 49-52, wherein the enzyme is a horseradish peroxidase (HRP).

Clause 55. The method of clause 54, wherein the chromogenic substrate is 3,3′,5,5′-tetramethylbenzidine (TMB).

Clause 56. The method of any one of clauses 34-55, wherein steps (i) to (iii) are performed on a negative control sample having no detectable NAb.

Clause 57. The method of clause 38, wherein the predetermined cut-point is a level corresponding to a 1% false-positive rate.

Clause 58. The method of clause 38 or 57, wherein the predetermined cut-point is 0.91.

Clause 59. The method of any one of clauses 34-58, wherein the sensitivity of detecting the NAb is less than 500 ng/mL.

Clause 60. The method of any one of clauses 34-59, wherein the sensitivity of detecting the NAb is less than 250 ng/mL.

Clause 61. The method of any one of clauses 34-60, wherein the sensitivity of detecting the NAb is less than 150 ng/mL.

Clause 62. The method of any one of clauses 34-61, wherein a target tolerance is about 1000 ng/mL.

Clause 63. The method of any one of clauses 34-62, wherein the target antibody comprises SEQ ID NOs: 2 and 5.

Clause 64. The method of any one of clauses 34-63, wherein the target antibody comprises SEQ ID NOs: 1 and 4.

Clause 65. A kit for detecting a NAb against a target antibody comprising in a sample comprising: (i) an affinity antibody comprising the target antibody conjugated to a first affinity label; (ii) a first solid phase coated with a second affinity label; (iii) a detection antibody comprising the target antibody conjugated to a detection label; (iv) a substrate; and (v) TNFα, wherein the target antibody comprises an anti-TNFα chimeric human-murine monoclonal IgG1-kappa antibody comprising SEQ ID NO: 9-11 or 15-17 and SEQ ID NOs: 12-14 or 18-20.

Clause 66. The kit of clause 65, wherein the first affinity label is biotin.

Clause 67. The kit of clause 65 or 66, wherein the second affinity label is streptavidin.

Clause 68. The kit of any one of clauses 65-67, wherein the detection label is a luminescent compound, an enzyme configured to convert a chromogenic substrate into a pigment, or biotin.

Clause 69. The kit of any one of clauses 65-68, wherein the first solid phase is a microbead.

Clause 70. The kit of any one of clauses 65-69, wherein the first solid phase is a magnetic microbead.

Clause 71. The kit of any one of clauses 65-70, wherein the detection label is biotin.

Clause 72. The kit of any one of clauses 65-71, further comprising an acid wash buffer.

Clause 73. The kit of any one of clauses 65-72, further comprising a washing buffer.

Clause 74. The kit of any one of clauses 65-73, further comprising an elution buffer. Claims group

We claim:

1. A method for detecting a neutralizing antibody (Nab) against a target antibody in a sample comprising:
(i) disassociating the NAb from the target antibody by adding an acid wash to the sample;
(ii) purifying the NAb in the sample;
(iii) contacting the purified NAb with a detection antibody comprising an anti-TNFα antibody conjugated to a detection label;
(iv) contacting the purified NAb and the detection antibody with a TNFα-coated substrate; and
(v) detecting the NAb when the detection antibody binds the NAb instead of the TNFα-coated substrate,
wherein the target antibody is an anti-TNFα chimeric human-murine monoclonal IgG1-kappa antibody comprising SEQ ID NO: 9-11 or 15-17 and SEQ ID NOs: 12-14 or 18-20.

2. The method of claim 1, further comprising detecting the detection label before detecting the NAb.

3. The method of claim 1, further comprising washing the TNFα-coated substrate before detecting the NAb.

4. The method of claim 1, wherein the acid wash comprises acetic acid.

5. The method of claim 1 or 4, wherein purifying the NAb of step (ii) comprises:
(a) contacting the sample with an affinity antibody comprising anti-TNFα antibody conjugated to a first affinity label;
(b) contacting the sample with a first solid phase coated with a second affinity label, wherein the first affinity label binds to the second affinity label;
(c) washing the first solid phase; and
(d) eluting the NAb from the first solid phase.

6. The method of claim 5, wherein the first affinity label is biotin.

7. The method of claim 5 or 6, wherein the first solid phase is a microbead.

8. The method of any one of claims 5-7, wherein the first solid phase is a magnetic microbead.

9. The method of any one of claims 5-8, wherein the second affinity label is streptavidin.

10. The method of any one of claims 1-9, wherein the NAb is purified using an automated purification system.

11. The method of any one of claims 1-9, wherein the detection label comprises a luminescent compound.

12. The method of any one of claims 1-9, wherein the detection label comprises an enzyme configured to convert a chromogenic substrate into a pigment.

13. The method of any one of claims 1-9, wherein the detection label is a third affinity label.

14. The method of claim 13, wherein detecting the detection label further comprises contacting the third affinity label with a fourth affinity label conjugated to a luminescent compound or enzyme configured to convert a chromogenic substrate into a pigment, wherein the third affinity label binds to the fourth affinity label.

15. The method of claim 14, wherein the third affinity label is biotin, and the fourth affinity label is streptavidin.

16. The method of claim 11 or 14, wherein detecting the detection label comprises detecting the luminescent compound.

17. The method of claim 12 or 14, wherein detecting the detection label comprises converting the chromogenic substrate into the pigment and detecting the pigment.

18. The method of claim 12 or 14, wherein detecting the detection label comprises converting the chromogenic substrate into the pigment and detecting an optical density of the sample.

19. The method of any one of claim 12 or 14-18, wherein the enzyme is a horseradish peroxidase (HRP).

20. The method of claim 19, wherein the chromogenic substrate is 3,3′,5,5′-tetramethylbenzidine (TMB).

21. The method of any one of claims 1-20, wherein steps (i) to (v) are performed on a negative control sample having no detectable NAb.

22. The method of any of claims 1-21, wherein the detection antibody binding the NAb instead of the TNFα-coated substrate is detected when the detection label is present at a level below a predetermined level.

23. The method of claim 22, wherein the detection label is present at the level below the predetermined level when an optical density of the sample is above a predetermined cut-point.

24. The method of claim 23, wherein the predetermined cut-point is the level corresponding to a false-positive rate of 3% or less.

25. The method of any one of claims 23-24, wherein the predetermined cut-point is the level corresponding to a 1% false-positive rate.

26. The method of any one of claims 23-25, wherein the predetermined cut-point is 0.5-2.

27. The method of claim 26, wherein the predetermined cut-point is 0.91.

28. The method of any one of claims 1-27, wherein a sensitivity of detecting the NAb is less than 500 ng/mL.

29. The method of claim 28, wherein the sensitivity of detecting the NAb is less than 250 ng/mL.

30. The method of any one of claim 28 or 29, wherein the sensitivity of detecting the NAb is less than 150 ng/mL.

31. The method of any one of claims 1-30, wherein a target tolerance is about 1000 ng/mL.

32. The method of any one of claims 1-31, wherein the target antibody comprises SEQ ID NOs: 2 and 5.

33. The method of any one of claims 1-32, wherein the target antibody comprises SEQ ID NOs: 1 and 4.

34. A method for detecting an NAb against a target antibody in a sample comprising:
(i) purifying the NAb in the sample;
(ii) determining whether the NAb is present in the sample by performing a competitive binding assay, wherein the NAb is present if the NAb binds the anti-TNFα antibody with a higher affinity than the affinity of the anti-TNFα antibody to TNFα; and
(iii) detecting the NAb;
wherein the target antibody comprises an anti-TNFα chimeric human-murine monoclonal IgG1-kappa antibody comprising SEQ ID NO: 9-11 or 15-17 and SEQ ID NOs: 12-14 or 18-20, and
wherein a sensitivity of the method is 250 ng/mL or less.

35. The method of claim 34, wherein before the NAb is purified, the method further comprises disassociating the NAb from the target antibody by adding an acid wash to the sample.

36. The method of claim 34 or 35, wherein the competitive binding assay comprises:
(a) contacting the purified NAb with a detection antibody comprising an anti-TNFα antibody conjugated to a detection label;
(b) contacting the purified NAb and the detection antibody with a TNFα-coated substrate;
(c) washing the TNFα-coated substrate; and
(d) detecting the detection label.

37. The method of claim 36, wherein detecting the NAb comprises determining whether the detection label is present at a level below a predetermined level.

38. The method of claim 37, wherein the detection label is present at the level below the predetermined level when an optical density is above a predetermined cut-point.

39. The method of any one of claims 35-38, wherein the acid wash comprises acetic acid.

40. The method of any one of claims 34-39, wherein purifying the NAb comprises:
(a) contacting the sample with an affinity antibody comprising an anti-TNFα antibody conjugated to a first affinity label;
(b) contacting the sample with a first solid phase coated with a second affinity label, wherein the first affinity label binds to the second affinity label;
(c) washing the first solid phase; and
(d) eluting the NAb from the first solid phase.

41. The method of claim 40, wherein the first affinity label is biotin.

42. The method of claim 40 or 41, wherein the first solid phase is a microbead.

43. The method of any one of claims 40-42, wherein the first solid phase is a magnetic microbead.

44. The method of any one of claims 40-43, wherein the second affinity label is streptavidin.

45. The method of any one of claims 34-44, wherein the NAb is purified using an automated purification system.

46. The method of any one of claims 36-45, wherein the detection label comprises a luminescent compound.

47. The method of any one of claims 36-45, wherein the detection label comprises an enzyme configured to convert a chromogenic substrate into a pigment.

48. The method of any one of claims 36-45, wherein the detection label is a third affinity label.

49. The method of claim 48, wherein detecting the detection label further comprises contacting the third affinity label with a fourth affinity label conjugated to a luminescent compound or enzyme configured to convert a chromogenic substrate into a pigment, wherein the third affinity label binds to the fourth affinity label.

50. The method of claim 49, wherein the third affinity label is biotin, and the fourth affinity label is streptavidin.

51. The method of claim 46 or 49, wherein detecting the detection label comprises detecting the luminescent compound.

52. The method of claim 47 or 49, wherein detecting the detection label comprises converting the chromogenic substrate into the pigment and detecting the pigment.

53. The method of claim 47 or 49, wherein detecting the detection label comprises converting the chromogenic substrate into the pigment and detecting an optical density of the sample.

54. The method of any one of claim 47 or 49-52, wherein the enzyme is a horseradish peroxidase (HRP).

55. The method of claim 54, wherein the chromogenic substrate is 3,3′,5,5′-tetramethylbenzidine (TMB).

56. The method of any one of claims 34-55, wherein steps (i) to (iii) are performed on a negative control sample having no detectable NAb.

57. The method of claim 38, wherein the predetermined cut-point is a level corresponding to a 1% false-positive rate.

58. The method of claim 38 or 57, wherein the predetermined cut-point is 0.91.

59. The method of any one of claims 34-58, wherein the sensitivity of detecting the NAb is less than 500 ng/mL.

60. The method of any one of claims 34-59, wherein the sensitivity of detecting the NAb is less than 250 ng/mL.

61. The method of any one of claims 34-60, wherein the sensitivity of detecting the NAb is less than 150 ng/mL.

62. The method of any one of claims 34-61, wherein a target tolerance is about 1000 ng/mL.

63. The method of any one of claims 34-62, wherein the target antibody comprises SEQ ID NOs: 2 and 5.

64. The method of any one of claims 34-63, wherein the target antibody comprises SEQ ID NOs: 1 and 4.

65. A kit for detecting a NAb against a target antibody in a sample comprising:
(i) an affinity antibody comprising the target antibody conjugated to a first affinity label;
(ii) a first solid phase coated with a second affinity label;
(iii) a detection antibody comprising the target antibody conjugated to a detection label;
(iv) a substrate; and
(v) TNFα,
wherein the target antibody is an anti-TNFα chimeric human-murine monoclonal IgG1-kappa antibody comprising SEQ ID NO: 9-11 or 15-17 and SEQ ID NOs: 12-14 or 18-20.

66. The kit of claim 65, wherein the first affinity label is biotin.

67. The kit of claim 65 or 66, wherein the second affinity label is streptavidin.

68. The kit of any one of claims 65-67, wherein the detection label is a luminescent compound, an enzyme configured to convert a chromogenic substrate into a pigment, or biotin.

69. The kit of any one of claims 65-68, wherein the first solid phase is a microbead.

70. The kit of any one of claims 65-69, wherein the first solid phase is a magnetic microbead.

71. The kit of any one of claims 65-70, wherein the detection label is biotin.

72. The kit of any one of claims 65-71, further comprising an acid wash buffer.

73. The kit of any one of claims 65-72, further comprising a washing buffer.

74. The kit of any one of claims 65-73, further comprising an elution buffer.


SEQUENCE LISTING (20 sequences)

SEQ ID NO: 1 - protein, 450 aa, synthetic construct
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 118 aa, synthetic construct
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTV

SEQ ID NO: 3 - protein, 332 aa, synthetic construct
SSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, synthetic construct
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 106 aa, synthetic construct
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEV

SEQ ID NO: 6 - protein, 108 aa, synthetic construct
KRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 718 bp, synthetic construct
  source 1..718: mol_type = other DNA
gctagcgaattccaccatgggctggtcctgcatcatcctgtttctggtggccaccgccaccggcgtgcactccgacatcctgctgacccagtcccccgccatcctgtccgtgtctcccggcgagcgggtgtccttctcctgccgggcctcccagttcgtgggctcctccatccactggtatcagcagcggaccaacggctcccctcggctgctgatcaagtacgcctccgagtccatgtccggcatcccttcccggttctccggctccggctctggcaccgacttcaccctgtccatcaacaccgtggagtccgaggatattgccgactactactgccagcagtcccactcctggcctttcaccttcggctccggcaccaacctggaggtgaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 8 - DNA, 1451 bp, synthetic construct
  source 1..1451: mol_type = other DNA
gctagcgatatcgaattcttttcttttgttttgtttccaccatgggctggtcctgcatcatcctgtttctggtggccaccgccaccggcgtgcactccgaggtgaagctggaggagtctggcggcggactggtgcagcctggcggctccatgaagctgtcctgcgtggcctccggcttcatcttctccaaccactggatgaactgggtgcgccagtcccctgagaagggcctggagtgggtggccgagatccggtccaagtccatcaactccgccacccactacgccgagtccgtgaagggccggttcaccatctcccgggacgactccaagtccgccgtgtacctgcagatgaccgacctgcggaccgaggacaccggagtgtactactgctcccggaactactacggctccacctacgactactggggccagggcaccacactgaccgtgagctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagtaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 9 - protein, 8 aa, synthetic construct
GFIFSNHW

SEQ ID NO: 10 - protein, 10 aa, synthetic construct
IRSKSINSAT

SEQ ID NO: 11 - protein, 11 aa, synthetic construct
SRNYYGSTYDY

SEQ ID NO: 12 - protein, 6 aa, synthetic construct
QFVGSS

SEQ ID NO: 13 - skipped

SEQ ID NO: 14 - protein, 9 aa, synthetic construct
QQSHSWPFT

SEQ ID NO: 15 - protein, 5 aa, synthetic construct
NHWMN

SEQ ID NO: 16 - protein, 19 aa, synthetic construct
EIRSKSINSATHYAESVKG

SEQ ID NO: 17 - protein, 9 aa, synthetic construct
NYYGSTYDY

SEQ ID NO: 18 - protein, 11 aa, synthetic construct
RASQFVGSSIH

SEQ ID NO: 19 - protein, 7 aa, synthetic construct
YASESMS

SEQ ID NO: 20 - protein, 9 aa, synthetic construct
QQSHSWPFT